SEQUENCE LISTING (1 sequence)

SEQ ID NO: 1 - protein, 40 aa, Homo sapiens
DAEFRHDSGYEVHHQKLVFFAEDVGSNKGAIIGLMVGGVV

(19) United States
(12) Patent Application Publication
(10) Pub. No.: US 2025/0338875 A1
(43) Pub. Date: Nov. 6, 2025

(54) NICKEL CHELATION THERAPY

(71) Applicant: UNIVERSITY OF GEORGIA RESEARCH FOUNDATION, INC., Athens, GA (US)

(72) Inventors: Robert J. Maier, Athens, GA (US); Stéphane Benoit, Bishop, GA (US)

(21) Appl. No.: 19/272,261

(22) Filed: Jul. 17, 2025

Related U.S. Application Data
(62) Division of application No. 17/514,133, filed on Oct. 29, 2021.
Application No. 17/514,133 is a continuation-in-part of application No. PCT/US2020/030483, filed on Apr. 29, 2020.
(60) Provisional application No. 62/840,543, filed on Apr. 30, 2019.

(51) Int. Cl. A23K 20/20; A23K 20/105; A23K 50/75; A61K 33/34

(52) U.S. Cl. CPC A23K 20/20; A23K 20/105; A23K 50/75; A61K 33/34

(57) ABSTRACT

This disclosure describes compositions including dimethylglyoxime (DMG) and methods of using those compositions including, for example, to reduce the availability of nickel in the subject. In some aspects, the composition may be administered to a subject suffering from or susceptible to a bacterial infection and/or to a subject suffering from or susceptible to a amyloid-β peptide aggregation. In some aspects, this disclosure describes using DMG or a composition including DMG to disrupt a biofilm or reduce the likelihood of biofilm formation.

### CONTINUING APPLICATION DATA

This application is a continuation in part of PCT/US2020/030483, filed on Apr. 29, 2020, which claims the benefit of U.S. Provisional Application Ser. No. 62/840,543, filed Apr. 30, 2019, the disclosures of which are incorporated by reference herein in their entireties.

### GOVERNMENT FUNDING

This invention was made with government support under AI121181 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “0235-000285US20_ST25.txt” having a size of 1 Kb and created on Oct. 27, 2021. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD

The present disclosure generally relates to metal chelator compounds, compositions containing the same, and methods of using the compounds.

### BACKGROUND

Enterobacteriaceae illnesses, including those caused by Escherichia, Klebsiella, Salmonella, Shigella, and Yersinia species, cost billions of dollars in diarrheal illness treatment and lead to millions of human deaths every year. For instance, in 2013, the annual cost associated with non-typhoidal Salmonella infections alone was estimated at 3.67 billion dollars in the United States.

Among Enterobacteriaceae, multi-drug resistant (MDR) species pose one of the biggest public health challenges of our time. A recent study conducted over three years in a French hospital found that bloodstream infections with MDR Enterobacteriaceae accounted for more than 70 percent of all bloodstream infections with MDR bacterial strains. MDR bacterial strains include, for example, extended-spectrum β-lactamase (ESBL)-producing and carbapenem-resistant Enterobacteriaceae (CRE). Resistance to drugs can emerge rapidly, and responses to these emerging public threats are slow or even nonexistent. New avenues to disable these and related pathogens would be advantageous.

Additionally, Alzheimer's disease (AD), discovered more than a century ago by Lois Alzheimer (Alzheimer Allgemeine Zeitschrift fur Psychiatrie und Psychisch-Gerichtlich Medizin 64, 146-148 (1907)), is the most common cause of dementia in elderly people, as well as in individuals with Down syndrome who survive beyond age 50. AD is a major health problem in the United States and the rest of the world. According to the most recent national vital statistics report available in the USA (year 2017), AD is estimated to be the fifth leading cause of death for people aged 65 and over, and the third leading cause of death for people aged 85 and over, behind heart disease and cancer (Kochanek et al., (2019)). In the absence of a cure, and because the population is rapidly aging, a study from the Alzheimer's Association predicts that by mid-century 13.8 million Americans will live with the disease, with one new case of AD developing every 33 seconds, resulting in nearly one million new cases per year.

Based on the time of onset, AD is classified into two types: early-onset AD (EOAD), which typically develops before the age of 65, and late-onset AD (LOAD) for those older than 65 (Zetterberg Lancet 368, 387 (2006)). In addition to intraneuronal tangles of hyperphosphorylated tau (τ) protein (Jebarupa et al. Biophys Chem 241, 27-37 (2018)), one hallmark of AD is characterized by various pathological markers in the brain, including accumulation of Amyloid Beta (Aβ) protein (in the form of senile plaques), as first proposed by Hardy and Higgins in a landmark study known as “the amyloid beta cascade hypothesis” (Hardy et al. Science 256, 184+(1992)). Sequential proteolysis of the amyloid precursor protein (APP), an ancient and highly conserved protein (Tharp et al. BMC genomics 14, 290 (2013)), by β-secretase and γ-secretase enzymes yields Aβ peptides of various lengths (38, 40 or 42 amino acids), depending upon the exact site of cleavage (Haass et al. Cell 75, 1039-1042 (1993)). While the most abundant Aβ peptide is Aβ₄₀, the most toxic is Aβ₄₂ (Galante et al. Int J Biochem CellBiol 44, 2085-2093 (2012)). The release of Aβ peptides is a normal physiological process. For example, Aβ peptides are naturally present in both the brain and the cerebrospinal fluid throughout the life of an individual (Seubert et al. Nature 359, 325-327 (1992); Vigo-Pelfrey et al. J Neurochem 61, 1965-1968 (1993); Ida et al. J Biol Chem 271, 22908-22914 (1996)) and they are also produced by cultured cells during normal metabolism (Haass et al. Nature 359, 322-325 (1992)). However, once Aβ peptides form filamentous aggregates (e.g., amyloids), not only can they propagate their abnormal structures to the same precursor molecules (seeding), they can also propagate to other protein monomers (cross-seeding), such as those involved in Parkinson's or Type 2 diabetes diseases (Ivanova et al. Biophys Chem 269, 106507 (2021)). New avenues to reduce the likelihood and/or break up Aβ peptides amyloids would be advantageous.

### SUMMARY OF THE APPLICATION

Provided herein are compositions that include a chelator and a carrier, and methods of use thereof. In one embodiment, the chelator is dimethylglyoxime (DMG). In one embodiment, the carrier is a pharmaceutically acceptable carrier. In one embodiment, the carrier includes a liquid elixir. In one embodiment, the liquid elixir includes a sugar. In one embodiment, the carrier includes a food product. In one embodiment, the composition includes an additional active agent. In one embodiment, the additional active agent includes a metallic ion or a compound that produces a metallic ion. In one embodiment, the additional active agent includes a divalent cation.

In one embodiment, the composition of any one of the previous embodiments is administered to a subject. In one embodiment, the subject is a human or an animal. In one embodiment, the subject is a chicken. In one embodiment the composition includes copper.

In one embodiment, the composition of any of the previous embodiments is administered to a subject infected with a pathogen or susceptible to infection by a pathogen. In one embodiment, the pathogen includes a pathogen that has a nickel-containing enzyme, a fungus that has nickel-containing enzyme, or a non-fungal eukaryotic pathogen that has a nickel-containing enzyme, or combinations thereof. In one embodiment, the pathogen is a multi-drug resistant pathogen. In one embodiment the pathogen is Acinetobacter baumannii, Enterococcus faecium, Escherichia co/i, Helicobacter pylori, Haemophilus influenzae, Neisseria gonorrhoeae, Streptococcus pneumoniae, a Campylobacter species, an Enterobacter species, a Klebsiella species, a Morganella species, a Proteus species, a Providencia species, a Pseudomonas species, a Salmonella species, a Serratia species, a Shigella species, or a Staphylococcus species, or a combination thereof; Cryptococcus neoformans, Cryptococcus gattii, Coccidioides posadasii, Histoplasma capsulatum, or Paracoccidioides brasiliensis, or a combination thereof, or Pythium insidiosum, Leishmania major, Leishmania donovani, or Trypanosoma cruzi, or a combination thereof.

In one embodiment, the composition of any of the previous embodiments is administered to a subject that is suffering from or susceptible to a disease associated with amyloid-β peptide aggregation. In one embodiment, the disease is Alzheimer's, Down Syndrome, or both.

Also provided herein is a method of disrupting a biofilm or preventing biofilm formation. In one embodiment, the method includes treating a surface with dimethylglyoxime (DMG). In one embodiment, the biofilm includes a Campylobacter species, Helicobacter pylori, a Klebsiella species, a Proteus species, a Pseudomonas species, a Salmonella species, or a Staphylococcus species, or a combination thereof.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

The terms “comprises” and “variations” thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

As used herein, the term “infection” refers to the presence of and multiplication of a microbe in the body of a subject. The infection can be clinically inapparent, or result in symptoms associated with disease caused by the microbe. The infection can be at an early stage, or at a late stage. Examples of a microbe include a fungus and a bacterium.

As used herein, the term “Ni-containing enzyme” refers to a metalloenzyme catalyzes a reaction with at least one Ni atom cofactor. A Ni-containing enzyme may have additional metallic cofactors that are not Ni. A Ni-containing enzyme may have more than one Ni atom cofactor.

As used herein, the term “substantially free” of a particular compound means that the compositions of the present disclosure contain less than 1,000 parts per million (ppm) of the recited compound. The term “essentially free” of a particular compound means that the compositions of the present invention contain less than 100 parts per million (ppm) of the recited compound. The term “free” of a particular compound means that the compositions of the present invention contain less than 20 parts per billion (ppb) of the recited compound. In the context of the aforementioned phrases, the compositions of the present invention contain less than the aforementioned amount of the compound whether the compound itself is present in unreacted form or has been reacted with one or more other materials.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows the structure of dimethylglyoxime (DMG) and DMG-Ni. Two molecules of DMG are needed to coordinate one molecule of Ni²⁺. FIG. 1B shows disodium salt octahydrate DMG.

FIG. 2 shows the effect of DMG on the growth of multi-drug resistant (MDR) pathogens. K. pneumoniae and S. Typhimurium strains. K. pneumoniae BAA2472 (white bars), S. Typhimurium 700408 (black bars), and S. Typhimurium 14028 (grey bars) were inoculated (approximately 5×10⁶ CFU/mL) in appropriate media containing increasing concentrations of DMG, as indicated, and grown at 37° C. under aerobic conditions and constant shaking. Cell growth (CFU/mL) at 16 hours was determined by serial dilution and plating. Results shown represent means and standard deviations from three biological replicates. An asterisk above a bar indicates the bacterial growth (CFU/mL) was significantly lower compared to the no DMG control (P<0.01, Student's t-test).

FIG. 3A-FIG. 3B show regions of 800 MHz proton-carbon correlated spectra showing diagnostic DMG signals in liver extracts from DMG-fed mice (FIG. 3A) and no-DMG control mice (FIG. 3B). FIG. 3A shows one-bond correlated methyl protons and methyl carbon signal in an HSQC NMR spectrum (top panel) and two-bond correlated methyl protons to oxime carbon signal (bottom panel). FIG. 3B shows HSQC NMR spectrum corresponding to the top panel of FIG. 3A.

FIG. 4A-FIG. 4B show DMG-chelation attenuates S. Typhimurium 14028 virulence in mice. Mouse survival following infection with S. Typhimurium 14028 and treatment with DMG (white circles) or no DMG treatment (black circles). For DMG-treated mice, a dose of 3 mg DMG (in water) was orally given six hours after infection with S. Typhimurium and then once daily, until day seven (FIG. 4A) or until day nine (FIG. 4B) post-inoculation. The last day of DMG treatment is indicated by an arrow, and the number of mice (n) used for each experiment is shown in the upper right box.

FIG. 5 shows DMG treatment decreases S. Typhimurium organ burden in mice. Organ colonization of S. Typhimurium strain 14028 in the livers (circles) and spleens (diamonds) of infected mice (72 hours post S. Typhimurium inoculation), after treatment with DMG (white symbols) or no DMG treatment (black symbols). Each symbol represents the mean (Log₁₀) CFU/mL for one organ (liver or spleen, as indicated) and each horizontal bar represents the geometric mean of the colonization load for each group. The organ burden (mean colonization) in the DMG-treated group is significantly lower compared to the control group (no DMG), P<0.01 for livers and P<0.025 for spleens, respectively.

FIG. 6 shows DMG-treatment of MDR K. pneumoniae and MDR S. Typhimurium attenuates virulence in the Galleria mellonella insect model. G. mellonella larvae (n=10 for each condition) were inoculated with 5 μL of the following: 0.8% NaCl (control), white squares; 250 mM DMG, black diamonds; 5×10⁵ CFUs K. pneumoniae BAA2472, black circles; 250 mM DMG (left proleg) and 5×10⁵ CFUs K. pneumoniae BAA2472 (right proleg), white circles; 5×10⁵ CFUs S. Typhimurium 14028, gray triangles; 250 mM DMG (left proleg) and 5×10⁵ CFUs S. Typhimurium 14028 (right proleg), white triangles.

FIG. 7 shows the time-dependent Aβ₄₀ peptide aggregation in absence or presence of Ni(II) and DMG. Aβ₄₀ peptide (40 μM) and thioflavin (40 μM) were mixed: in the absence of Ni(II) and DMG (white squares); with 100 μM Ni(II) (black squares); with 100 μM Ni(II) and 100 μM DMG (grey circles); with 100 μM Ni(II) and 500 μM DMG (black triangles); with 100 μM Ni(II) and 1 mM DMG (white circles). ThT-based fluorescence was measured every 5 min for 60 min. A ThT-only background control (no Aβ₄₀ peptide) was included in the assay (data not shown). Results shown for each time point represent the mean and standard deviation (error bars) of background-subtracted values for triplicate wells. Results shown here correspond to Example 2, Table 6.

FIG. 8 shows the time-dependent Aβ₄₀ peptide aggregation in the absence or presence of Ni(II) and DMG at various pH values. Aβ₄₀ peptide (25 μM) and thioflavin (40 μM) were mixed in absence of metal or DMG (circles), in presence 25 μM Ni(II) (squares), or in presence of 10 μM Zn(II) (triangles). The final pH in the reaction was 6.5 (white symbols), 7.5 (grey symbols), or 8.5 (black symbols). For example, black triangles represent RFUs measured in presence of Zn(II) at pH 8.5. ThT-based fluorescence was measured every 3 min for 120 min. A ThT-only background control (no Aβ₄₀ peptide) was included in the assay (data not shown). Results shown for each time point represent the mean and standard deviation (error bars) of background-subtracted values for triplicate wells.

FIG. 9 shows the isothermal titration calorimetry analysis of Ni binding to AP_{40⋅}. Top panel shows the raw data of heat release per injection, for 20 consecutive injections (2.38 μL) of NiSO₄ (1 mM) into a 500-μL cell containing AP₄₀ (20 μM). Bottom panel shows binding isotherms, obtained by integrating the areas of each injection peak. Data acquired with a NanoITC were analyzed using NanoAnalyze 1.2 software (TA Instruments). Shown in the inset are the best-fit values for the dissociation constant (K_{d}), stoichiometry (n), enthalpic change (ΔH), and entropic change (ΔS).

FIG. 10 shows a hypothetical model showing a dual role for nickel (Ni) and a proposed mode of action for DMG-mediated Ni chelation. Ni can bind to Aβ peptides, leading to aggregation and plaque formation (left side, metal hypothesis of AD). In addition, Ni is required as cofactor for enzymes (such as hydrogenase and urease) of pathogens previously shown to play a role in Aβ peptide aggregation (right side, infection hypothesis of AD). The Ni-chelator DMG could inhibit Aβ peptide aggregation and the progression of AD (crosses), either directly (left side) or indirectly (through pathogen inhibition, right side).

FIG. 11 shows the ICP-MS metal analysis of various kit components and proportion of metal brought by Aβ40 peptide in Example 4.

FIG. 12 shows the expected mass of various DMG-metal complexes for analysis with Fourier Transform Ion Cyclotron Resonance-Mass Spectrometry (FTICR-MS).

FIG. 13 shows the mass spectrum of an aqueous solution containing DMG (2Na, 8H₂O) and a simulation spectrum.

FIG. 14 shows the mass spectrum of an aqueous solution containing DMG (2Na, 8H₂O) and a simulation spectrum.

FIG. 15 shows the mass spectrum of an aqueous solution containing DMG (2Na, 8H₂O) with Ni and a simulation spectrum.

FIG. 16 shows the mass spectrum of an aqueous solution containing DMG (2Na, 8H₂O) and Ni, and a simulation spectrum.

FIG. 17 shows the mass spectrum of an aqueous solution containing DMG (2Na, 8H₂O) with Cu and a simulation spectra.

FIG. 18 shows an overlay of mass spectra of DMG with various metals.

FIG. 19 shows DMG inhibits exemplary biofilms. H. pylori 43504, S. Typhimurium 700408, and K. pneumoniae BAA2472 cells were incubated with DMG in 96 well plates for 48 hours (H. pylori 43504) or 18 hours (S. Typhimurium 700408, and K. pneumoniae BAA2472). Media only control contained only BHI-0.4% β-cyclodextrin (H. pylori 43504) or LB (S. Typhimurium 700408, and K. pneumoniae BAA2472). Determination of biofilm formation was measured by crystal violet staining. Error bars indicate standard deviation from one independent experiment with three to eight replicates per condition.

FIG. 20 shows the antibiofilm effect of DMG against an established H. pylori biofilm. H. pylori 43504 cells were incubated in 96 well plates for 48 hours to allow for biofilm formation.

DMG then was added to wells and incubated for an additional 24 hours. Determination of remaining biofilm was measured by crystal violet staining. Error bars indicate standard deviation from one experiment with 7-24 replicates per condition.

FIG. 21A-FIG. 21B show the effect of DMG alone, or in combination with CuSO₄, on the growth of Campylobacter concisus (FIG. 21A) or Campylobacter jejuni (FIG. 21), as described in Example 4. C. concisus or C. jejuni cells were harvested, standardized to OD₆₀₀ of one, and serially (10-fold) diluted in sterile 0.8% NaCl, before being spotted (5 μL) on solid media containing various concentrations of DMG or/and CuSO₄. Colony-forming units (CFUs) were counted after 24 hours incubation at 37° C. under microaerobic conditions (for C. jejuni) or hydrogen-enriched microaerobic conditions (for C. concisus).

FIG. 22 shows the timeline and conditions for a C. jejuni-chicken colonization experiment.

FIG. 23A shows a model for linking nickel and copper homeostasis in C. jejuni. FIG. 23B shows a proposed mode of action for DMG.

### DETAILED DESCRIPTION OF ILLUSTRATIVE EMBODIMENTS

This disclosure describes compositions including dimethylglyoxime (DMG) and methods of using those compositions. In some aspects, this disclosure describes administering the composition including DMG to reduce the availability of nickel in the subject. In one aspect, this disclosure describes administering the composition to a subject suffering from or susceptible to a bacterial infection. In some embodiments, the bacterial infection may include a multi-drug resistant (for example, an antibiotic resistant) bacterium. In another aspect, this disclosure describes administering a composition including DMG to a subject suffering from or susceptible to a amyloid-β peptide aggregation. In a further aspect, this disclosure describes administering a composition including DMG to a subject suffering from or susceptible to a nickel allergy and/or an obese subject. In yet another aspect, this disclosure describes administering a composition including DMG to a subject to alter the balance of bacteria in the subject's microbiome. In some aspects, this disclosure describes using DMG or a composition including DMG to disrupt a biofilm or reduce the reduce likelihood of biofilm formation.

### Metal Chelation and Bacteria

Nickel is required as a cofactor for several bacterial enzymes, including acireductone dioxygenase, [NiFe]-hydrogenase, glyoxalase I, superoxide dismutase, and urease (Benoit and Maier 2013 Nickel Ions in Biological Systems, p. 1501-1505 in Kretsinger et al. (eds.), Encyclopedia of Metalloproteins. Springer New York, New York, NY). The nickel requirement for enzymes is associated only with bacterial (and not host) enzymes. Therefore, nickel sequestration is a possible therapeutic target to combat several pathogens (see, for example, Rowinska-Zyrek et al. 2014. Dalton Trans 43:8976-8989); see also Table 1A-Table 1B. For instance, targeting nickel trafficking pathways to inactivate both the H₂-uptake [Ni—Fe]hydrogenase and the urease in the gastric pathogen Helicobacter pylori has been proposed (de Reuse et al. 2013 Front Cell Infect Microbiol 3:94; Maier 2003 Microbes Infect. 5:1159-1163).

TABLE 1A

| | | Ni-containing enzyme | | | |
|---|---|---|---|---|---|
| | | Hydrogenase | Urease | Glyoxalase I | Aci. Dioxygenase |
| | | Uses or produces | Converts | detoxifies | converts 1,2-dihydroxy-5 |
| | | H₂ (number in | urea to | methyl-glyoxal | [symbol] |
| 2017 WHO priority MDR pathogens | | brackets → number | CO₂ | (metabolism | to 3-(methyl [symbol] |
| with Ni-containing enzymes | | of H₂ases | and NH₂ | byproduct) | [symbol] and CO₂ |
| Priority 1: CRITICAL | | | | | |
| Carbapenem [symbol] | Acinetobacter bo [symbol] mannii | | ✓ | ✓ | ✓ |
| Carbapenem [symbol] | Pseudomonas aeruginosa | ✓ | ✓ | ✓ | ✓ |
| Carbapenem [symbol] | Klebsiella pneumoniae | | ✓ | ✓ | ✓ |
| 3rd gen. Cephalosporin [symbol] | Escherichia coli | ✓ (4) | | ✓ | ✓ |
| Enterobacteriaceae | Enterobacter spp. | ✓ (2) | ✓ | ✓ | ✓ |
| | Serr [symbol] tia spp. | | ✓ | ✓ | ✓ |
| | Proteus spp. | ✓ | ✓ | ✓ | ✓ |
| | Providencia spp. | ✓ | ✓ | ✓ | ✓ |
| | Morganella spp. | ✓ | ✓ | ✓ | |
| Priority 2: HIGH | | | | | |
| Vancomycin [symbol] | Enterococcus faecium | | ✓ | | |
| Methycillin [symbol] , | Staphylococcus aureus | | ✓ | | |
| Vancomycin [symbol] | | | | | |
| Clarythromycin [symbol] | Helicobacter pylori | ✓ | ✓ | | |
| Fluroquinolone [symbol] | Campylobacter spp. | ✓ | | | |
| Fluroquinolone [symbol] | Salmonella spp. | ✓ (4) | | ✓ | ✓ |
| Cephalosporin [symbol] , | Neisseria gonorrhoeae | | ✓? | ✓ | |
| Fluroquinolone [symbol] | | | | | |
| Priority 3: MEDIUM | | | | | |
| Penicillin [symbol] | Streptococcus pneumoniae | | ✓ | ✓ | ✓ |
| Ampicillin [symbol] | Haemophilus influenzae | | ✓ | ✓ | |
| Fluroquinolone [symbol] | Shigella spp. | ✓ (3) | ✓ | ✓ | |

| | | Ni-containing enzyme |
|---|---|---|
| | | Comments |
| | | Hyp accessory proteins are needed for hydrogenase |
| | | Ure accessory proteins are needed for urease |
| 2017 WHO priority MDR pathogens | | GloA → glyoslase (135 [symbol] , very conserved) |
| with Ni-containing enzymes | | MtnD → acireductone dioxyenase |
| Priority 1: CRITICAL | | |
| Carbapenem [symbol] | Acinetobacter bo [symbol] mannii | Ure; Only one Hyp6 and one HycD homolog; |
| | | GloA; MntD |
| Carbapenem [symbol] | Pseudomonas aeruginosa | Hyp and Ure; GloA; MtnD |
| Carbapenem [symbol] | Klebsiella pneumoniae | Ure; GloA; MtnD |
| 3rd gen. Cephalosporin [symbol] | Escherichia coli | Hyp; GloA; MntD |
| Enterobacteriaceae | Enterobacter spp. | Hyp and Ure; GloA; MntD |
| | Serr [symbol] tia spp. | Ure; GloA; MtnD |
| | Proteus spp. | Hyp and Ure; GloA; MtnD |
| | Providencia spp. | Hyp and Ure; GloA; MtnD |
| | Morganella spp. | Hyp and Ure; GloA |
| Priority 2: HIGH | | |
| Vancomycin [symbol] | Enterococcus faecium | Ure accessory and structural |
| Methycillin [symbol] , [symbol] | Staphylococcus aureus | Prot annotated as “soluble hydrogenase”, |
| Vancomycin | | no match. Ure. |
| Clarythromycin [symbol] | Helicobacter pylori | Hyp and Urea |
| Fluroquinolone [symbol] | Campylobacter spp. | Hyp |
| Fluroquinolone [symbol] | Salmonella spp. | Hyp; GloA; MntD |
| Cephalosporin [symbol] , | Neisseria gonorrhoeae | UreA8C in Neisseria ssp. |
| Fluroquinolone [symbol] | | (can't find hit in N. gonnorrhea); GloA |
| Priority 3: MEDIUM | | |
| Penicillin [symbol] | Streptococcus pneumoniae | Hy [symbol] 8 homolog; Ure accessory; GloA; MntD |
| Ampicillin [symbol] | Haemophilus influenzae | Ure accessory found; GloA |
| Fluroquinolone [symbol] | Shigella spp. | Hyp and Ure accessory found; GloA |

[symbol]

indicates data missing or illegible when filed

TABLE 1B

| Pathogen | Ni-Enzyme |
|---|---|
| EUKARYOTES | |
| Human fungi | |
| Cryptococcus neoformans | Ure |
| Cryptococcus gattii | Ure |
| Coccidioides posadasii | Ure |
| Histoplasma capsulatum | Ure |
| Paracoccidioides brasiliensis | Ure |
| Oomycetes | |
| Pythium insidiosum | Ure |
| Protists | |
| Leishmania major | Glo-I |
| Leishmania donovani | Glo-I |
| Trypanosoma cruzi | Ard, Glo-I |
| PROKARYOTES | |
| Actinobacteria | |
| Actinomyces naeslundii | Ure |
| Corynebacterium urealyticum | Ure |
| Mycobacterium tuberculosis | Hyc, Ure |
| Streptomyces scabies | Sod |
| Firmicutes | |
| Clostridia | Glo-I |
| Staphylococcus aureus | Ure |
| Staphylococcus epidermidis | Ure |
| Staphylococcus saprophyticus | Ure |
| Streptococcus salivarius | Ure |
| Mollicutes | |
| Ureaplasma urealyticum | Ure |
| Ureaplasma parvum | Ure |
| Ureaplasma diversum | Ure |
| Proteobacteria | |
| Alphaproteobacteria | |
| Brucella abortus | Ure |
| Brucella melitensis | Ure |
| Brucella suis | Ure |
| Betaproteobacteria | |
| Neisseria meningitides | Glo-I |
| Neisseria gonorrhoeae | Glo-I |
| Gammaproteobacteria | |
| All γ-proteobacteria | Ard |
| All γ-proteobacteria | Glo-I |
| Acinetobacter baumannii | Ure |
| Acinetobacter lwoffii | Ure |
| Actinobacillus | Ure |
| pleuropneumoniae | Hyd-1 |
| Escherichia coli | Hyd-2, Hyc |
| E. coli (Shiga-toxin producing) | Ure |
| Edwardsiella tarda | Hyd |
| Haemophilus influenzae | Ure |
| Klebsiella pneumoniae | Ure |
| Morganella morganii | Ure |
| Proteus mirabilis | Hyd, Ure |
| Providencia stuartii | Ure |
| Pseudomonas aeruginosa | Glo-I |
| Salmonella Typhimurium | Hyd-1, Hyd-2, Hyd-5, Hyc |
| Shigella flexneri | Hyd |
| Vibrio parahaemolyticus | Ure |
| Yersinia enterocolitica | Ure |
| Yersinia pestis | Glo-I |
| Deltaproteobacteria | |
| Bilophila wadsworthia | Hyd |
| Epsilonproteobacteria | |
| Campylobacter jejuni | Hyd |
| Campylobacter concisus | Hyd |
| Helicobacter hepaticus | Hyd, Ure |
| Helicobacter mustelae | Ure |
| Helicobacter pylori | Hyd, Ure |

(Abbreviations:
Ard: acireductone dioxygenase;
Glo-I: Glyoxalase I;
Hyc: H₂-evolving hydrogenase;
Hyd: H₂-uptake hydrogenase;
Sod: superoxide dismutase;
Ure: urease.)

The nickel requirement for Cryptococcus neoformans's urease has been identified as the fungus's “Achilles' heel” (Morrow et al. 2013 mBio 4(4):e00408-13). Furthermore, the host defense protein human calprotectin sequesters nickel away from two pathogens, Staphylococcus aureus and Klebsiella pneumoniae, subsequently inhibiting their respective urease activity in bacterial culture (Nakashige et al. 2017 J Am Chem Soc 139:8828-8836). Many Enterobacteriaceae depend on nickel as a cofactor for their hydrogenase and/or urease enzymes. Escherichia coli and Salmonella species, including S. enterica serovar Typhimurium (referred to herein as S. Typhimurium), possess several Ni-containing hydrogenases (but not urease), while Klebsiella species, such as K. pneumoniae, possess a urease, as well as several hydrogenases. Molecular hydrogen (H₂) use (by H₂-uptake [Ni—Fe]hydrogenases Hya, Hyb and Hyd) is play a role in for S. Typhimurium virulence (Maier et al. 2004 Infect Immun 72:6294-6299; Maier et al. 2014 PLoS One 9:e110187; Lamichhane-Khadka et al. 2015. Infect Immun 83:311-316). Although not formally demonstrated, H₂ metabolism may play a role in K. pneumoniae's virulence. These results and predictions highlight the potential for Ni-chelation as an antibacterial therapy (Maier and Benoit, 2019 Inorganics 2019; 7:80); Benoit et al., 2020 MMBR 00092-19).

### Metal Chelation and Amyloid Beta (a$) Peptides

Alzheimer's disease occurs sporadically in most cases; however, a sizable number of cases can be linked to mutations in various genes. For instance, mutations in the APP gene, or in genes encoding for enzymes involved in the APP processing (e.g. PSEN1 or PSEN2), are predominantly associated with early onset Alzheimer's Disease, whereas mutations in genes encoding for enzymes related to Aβ turnover, such as the apolipoprotein E (e.g APOE), are usually associated with late onset Alzheimer's Disease (Saunders et al. Neurology 43, 1467-1472 (1993); Giri et al. Clinical interventions in aging 11, 665 (2016)). Besides genetic factors, environmental factors have been shown to play a role in AD, as revealed by a study on twins (Gatz et al. Archives of general psychiatry 63, 168-174 (2006)). Environmental factors include toxic gases, such as CO, CO₂, SO₂ and NO₂ (Saranya et al. Biophys Chem 263, 106394 (2020)), or metals, several of which have been shown to play a role on Aβ aggregation, fibrillization, and toxicity, with potential implications on the progression of AD (Liu et al. Acc Chem Res 52, 2026-2035 (2019)). The list includes heavy metals, such as aluminum (Al) (Ricchelli et al. Cellular and Molecular Life Sciences CMLS 62, 1724-1733 (2005)), cadmium (Cd) (Notarachille et al. Biometals 27, 371-388 (2014)) and mercury (Hg) (Olivieri et al. Journal of neurochemistry 74, 231-236 (2000); Meleleo et al. Biophys Chem 266, 106453 (2020)). The list includes essential metals, such as copper (Cu) and zinc (Zn), and to a lesser extent, iron (Fe)(Kozlowski et al. Coordination Chemistry Reviews 256, 2129-2141 (2012)). The role of Cu(I), Cu(II), and Zn(II) has been well documented (Tõugu et al. Metallomics 3, 250-261 (2011); Mathys et al. Neurotoxicity of Metals, 199-216 (2017); Hureau et al. Biochimie 91, 1212-1217 (2009)). For example, both Aβ₄₀ and Aβ₄₂ peptides bind Cu(II) or Zn(II) with significant affinity in vitro, leading to Aβ aggregation (Ricchelli et al. Cellular and Molecular Life Sciences CMLS 62, 1724-1733 (2005); Bush et al. Science 265, 1464-1467 (1994); Chen et al. Inorganic chemistry 48, 5801-5809 (2009); Huang et al. J Biol Chem 272, 26464-26470 (1997); Atwood et al. J Biol Chem 273, 12817-12826 (1998)). Additionally, a similar effect was observed in vivo, leading to plaque build-up and toxicity in AD animal models, for instance with Cu(II) in rabbits (Sparks et al. Proc Natl Acad Sci USA 100, 11065-11069 (2003)), or with Zn(II) in mice (Lee et al. Proc Natl Acad Sci USA 99, 7705-7710 (2002)). Furthermore, post-mortem analysis revealed that respective Cu, Fe, and Zn levels in plaques of AD brains were 5.7, 2.8, and 3.1-fold higher compared to normal brains (Lovell et al. J Neurol Sci 158, 47-52 (1998)). Finally, accumulation of Cu and Zn are co-localized with Aβ peptide deposits (Miller et al. J Struct Biol 155, 30-37 (2006)). Taken together, these results have resulted in the theory known as the “metal hypothesis of AD,” that links metal homeostasis (especially that of Cu, Fe and Zn) and AD (Bush et al. Neurotherapeutics 5, 421-432 (2008)). Recent discoveries on Aβ peptides-lipid interactions have confirmed the importance of metals in the onset and progression of AD. For example, Aβ peptides can associate with cellular membranes(Sciacca et al. ACS Chemical Neuroscience 11, 4336-4350 (2020)), and AP-bound metals (especially Zn and Al). Additionally, Aβ peptides can blockade and disrupt Ca²⁺ channels, leading to neurotoxicity(Kotler et al. Chemical Society Reviews 43, 6692-6700 (2014)).

In contrast to Cu, Fe, and Zn, which are required cofactors for hundreds of enzymes and fairly abundant in animals and humans (Maret Metallomics 2, 117-125 (2010)), nickel (Ni) does not appear to be needed in mammals, as mammalian hosts do not contain known Ni-dependent enzymes (Maier et al. Inorganics 7, 80 (2019)). Furthermore, Ni levels are low, with less than 5 ppm (μg/g of ash) in most human organs, corresponding to less than 1% of the amount of Zn measured in the brain, heart, lung, or muscle, and less than 0.1% of the amount of Zn in the liver and kidney (Iyengar et al., (1978)). Even though Ni is rarely mentioned in association with Aβ peptides, a potential role for this transition metal should not be discarded.

### Use of Metal Chelators at the Time of the Disclosure

At the time of this disclosure, metal chelators are already used (or are under evaluation in clinical trials) as drugs to control various human diseases, including cardiovascular diseases and Alzheimer's disease. Oral chelation is currently used to treat the hepatocellular copper inherited disorder known as Wilson disease. Furthermore, metal chelators can also be used to neutralize metal toxicity (Aaseth et al. 2015 J Trace Elem Med Biol 31:260-266; Sears 2013 Scientific World Journal 2013:219840), including nickel toxicity. For example, the chelating agent sodium diethyldithiocarbamate (DCC) has been shown to be an effective drug against nickel carbonyl poisoning (Sunderman 1990 Ann Clin Lab Sci 20:12-21). Similarly, disulfiram, a compound which is eventually metabolized in two DCC molecules, is FDA-approved to treat nickel carbonyl poisoning. For some diseases there are clear benefits to chelation therapy, but in some cases the therapies have been met with mixed results (Mathew et al. 2017 Cardiovasc Drugs Ther 31:619-625; Roberts et al. 2017 Handb Clin Neurol 142:141-156; Aaseth et al. 2015 J Trace Elem Med Biol 31:260-266). For example, some chelating chemicals have associated toxic side effects (Aaseth et al. 2015 J Trace Elem Med Biol 31:260-266; Andersen et al. 2016 J Trace Elem Med Biol 38:74-80.) Therefore, the use of “old” chelators such as ethylenediamine tetraacetate (EDTA) and 2,3-dimercaptopropanol (BAL) is now restricted, due to their toxicity (Aaseth et al. 2015 J Trace Elem Med Biol 31:260-266). The use of disulfiram is also controversial, since it has been associated with elevated nickel levels in rat brains (Baselt et al. 1982. Res Commun Chem Pathol Pharmacol 38:113-124), as well as with hepatotoxicity in humans (Kaaber et al. 1979. Contact Dermatitis 5:221-228), and elevated nickel levels in the body fluids of patients with chronic alcoholism (Hopfer et al. 1987 Res Commun Chem Pathol Pharmacol 55:101-109).

Metal chelators have been investigated to inhibit Aβ peptide aggregation with mixed outcomes (Santos et al. Coordination Chemistry Reviews 327, 287-303 (2016)). Chelators, such as ethylene glycol-bis(β-aminoethyl ether)-N,N,N′,N′-tetraacetic acid (EGTA or egtazic acid), N,N,N′,N′-tetrakis(2-pyridyl-methyl) ethylene diamine (“tpen”), and bathocuproine solubilize Aβ plaques from post-mortem brain tissue (Cherny et al. J Biol Chem 274, 23223-23228 (1999)). The 8-hydroxyquinoline derivatives Clioquinol and BPT-2, two copper-zinc chelators, have shown promising results in vitro (Bush et al. Neurotherapeutics 5, 421-432 (2008); Cherny et al. Neuron 30, 665-676 (2001)). The ability of 8-hydroxyquinoline derivatives Clioquinol and BPT-2 to cross the blood-brain-barrier (BBB) have been tested in clinical trials; however, the results appear inconclusive (Sampson et al. Cochrane Database of Systematic Reviews, (2014)). In another unrelated clinical trial, the rate of decline of daily living skills was significantly reduced in AD patients given desferrioxamine (sometimes called deferoxamine) intramuscular twice daily for two years (Crapper McLachlan et al. Lancet 337, 1304-1308 (1991)). This effect was initially attributed to aluminum chelation, however desferrioxamine binds preferentially to iron (also copper and zinc, albeit with lower affinity); hence it is hard to draw firm conclusions about this trial. Alternative ways to target and modulate the toxicity of metal-bound (or metal-free) Aβ species include the use of (i) glycosylated polyphenols and their esterified derivatives, which present the advantage of using natural low toxicity compounds (Korshavn et al. Sci Rep 5, 17842 (2015)); (ii) synthetic flavonoids and amino-isoflavones, which have shown promising results towards targeting metal sites(DeToma et al. Chem Sci 5, 4851-4862 (2014)); (iii) small molecules, such as N¹,N¹-dimethyl-N⁴-(pyridin-2-ylmethyl)benzene-1,4-diamine (“L2-b”) and its derivatives (Lee et al. Chemistry 23, 2706-2715 (2017); Lee et al. J Am Chem Soc 136, 299-310 (2014)); (iv) β-sheet breakers, which are small peptides (five amino-acids long) effective in reducing the Aβ₁₋₄₀ aggregation, even in the presence of metal ions (Stellato et al. Biophys Chem 229, 110-114 (2017)).

### Dimethylglyoxime (DMG)

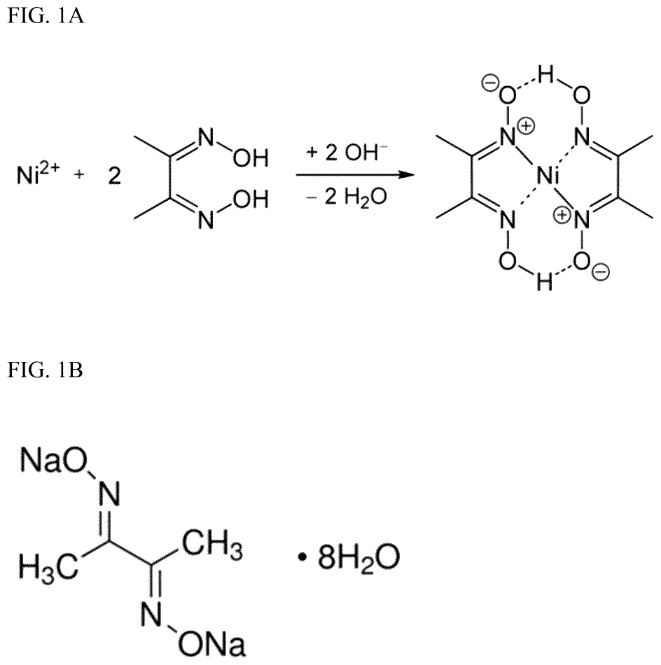

As further described in Example 1, nickel-specific chelation and the inhibition of bacterial growth was achieved in vitro and in vivo using dimethylglyoxime (DMG). Two molecules of DMG are needed to coordinate one Ni(II) molecule (FIG. 1A). DMG is reported to prefer forming a complex with nickel over other metals. The molecule was first described as nickel “precipitant” in 1946 (Minster 1946 Analyst 71:424-428) and was later used to identify nickel exposure of the skin (Choman 1962 Stain Technol 37:325-326), a procedure commonly known as “DMG test” (Thyssen et al. 2010. Contact Dermatitis 62:279-288; Julander et al. 2011 Contact Dermatitis 64:151-157).

DMG is also used to determine nickel levels in the environment (in soil, water, industrial effluents) (Ferancova et al. 2016 J Hazard Mater 306:50-57; Ershova et al. 2000 Fresenius J Anal Chem 367:210-211; Onikura et al. 2008 Environ Toxicol Chem 27:266-271). Additionally, DMG is used to assess possible toxic levels of nickel in various items, including jewelry (Thyssen et al. 2009. Sci Total Environ 407:5315-5318), mobile phones (Jensen et al. 2011. Contact Dermatitis 65:354-358) or surgical items (Boyd et al. 2018 Dermatol Online J 24(4)).

DMG has also been used to remove nickel from laboratory supplies, growth media, equipment, (Benoit et al. 2013 Infect Immun. 81:580-584) and from whole bacterial cells with the aim of studying roles of nickel in microbes. For example, DMG has been used in studies on maturation of Ni-binding proteins (for example, hydrogenase and/or urease) in H. pylori (Maier 2003 Microbes Infect 5:1159-1163; Seshadri et al. 2007 J Bacteriol 189:4120-4126; Saylor et al. 2018 Microbiology 164:1059-1068; Benanti et al. 2009 J Bacteriol 191:2405-2408) or in Azotobacter chroococcum (Partridge et al 1982 Biochem J 204:339-344).

In 2007, insoluble DMG (Sigma #D-1885 with formula weight of 116.12 g/mol) was tested for pathogen-inhibitory properties. The DMG was administered as ethanolic solutions, and the animals (BALB/C mice) appeared ill even when given chelator alone, exhibiting symptoms consistent with extreme constipation. Without wishing to be bound by theory, it is believed that upon stomach absorption of the ethanol, DMG came out of solution and caused intestinal compaction.

### Methods of Using DMG

In one aspect, this disclosure describes a method that includes administering a chelator or a pharmaceutical composition including a chelator to a subject. In some embodiments, the chelator preferably includes dimethylglyoxime (DMG).

In some embodiments, the chelator or the pharmaceutical composition including the chelator may be administered to a subject to reduce the availability of a metal species in the subject. In some embodiments, the metal species preferably includes nickel. In some embodiments, the metal species includes copper.

In some embodiments, the chelator may be administered to a subject in an amount sufficient to reduce the availability of a metal species in the subject. In some embodiments, the chelator may be administered (including, for example, to a subject) in an amount sufficient to inhibit the growth of a pathogen. In some embodiments, the chelator may be administered to a subject in an amount sufficient to halt or slow the progression of a pathogenic infection or symptoms of a pathogenic infection within the subject. In some embodiments, the chelator may be administered to a subject suffering from or susceptible to a disease associated with amyloid-β peptide aggregation, for example Alzheimer's disease or Down syndrome. In some embodiments, the chelator may be administered to a subject suffering from or susceptible to a nickel allergy. In some embodiments, the chelator may be administered to an obese subject. In some embodiments, the chelator may be administered to a subject to alter the balance of bacteria in the subject's microbiome.

In some embodiments, the DMG preferably includes soluble DMG. In some embodiments, soluble DMG preferably includes water soluble DMG. In some embodiments, the soluble DMG includes the disodium salt DMG and/or disodium salt octahydrate DMG. In some embodiments, the soluble DMG preferably includes the disodium salt octahydrate DMG. Given the previous results with insoluble DMG including the toxicity observed in mice, the high efficacy and low toxicity of DMG described herein was particularly unexpected. Solubility is based on the vendor's definition of solubility as can be ascertained by the vendor's technical data, the Material Safety Data Sheet, the Safety Data Sheet, or by other data provided by the vendor. Additionally, the solubility may be determined by dissolving an amount of DMG in a known amount of solvent (e.g., water) and visually observing the opacity of the resulting solution. Generally, DMG is not soluble if the resulting solution has visible precipitates or is opaque. Generally, DMG is soluble if the resulting solution is clear with no visible principates. Generally, anhydrous DMG is not soluble in aqueous buffers and requires an additional non-aqueous solvent to dissolve. Generally, the disodium salt octahydrate DMG is soluble in water and aqueous buffers.

A subject may include a human or an animal. An animal may include a companion animal, a domesticated animal such as a farm animal, an animal used for research, or an animal in the wild. Companion animals include, but are not limited to, dogs, cats, hamsters, gerbils, and guinea pigs. Domesticated animals include, but are not limited to, domesticated fowl including chickens and turkeys, cattle, horses, pigs, goats, and llamas. Research animals include, but are not limited to, mice, rats, dogs, apes, and monkeys.

In some embodiments, treatment of domesticated fowl, such as chickens or turkeys, with a chelator (for example, DMG) may be particularly desirable because infection of domesticated fowl with C. jejuni is a major cause of food poisoning. In some embodiments, DMG may be co-administered with copper in domesticated fowl. As described in Example 4 and Example 5, while millimolar levels of DMG are bacteriostatic against C. jejuni, the addition of micromolar levels of copper(II) surprisingly rendered millimolar levels of DMG bactericidal towards C. jejuni. Additionally, without wishing to be bound by theory, it is believed that, because of the high levels of copper already present in the diets of many chickens, co-administration of additional copper may not be necessary to see the bactericidal effect of DMG. In some embodiments, domesticated fowl, such as chickens, are treated with a chelator (for example, DMG) without additional providing additional copper beyond the copper already present in the diet of the chickens. Example 5 demonstrates that millimolar concentrations of DMG reduces the likelihood of C. jejuni colonization in chickens.

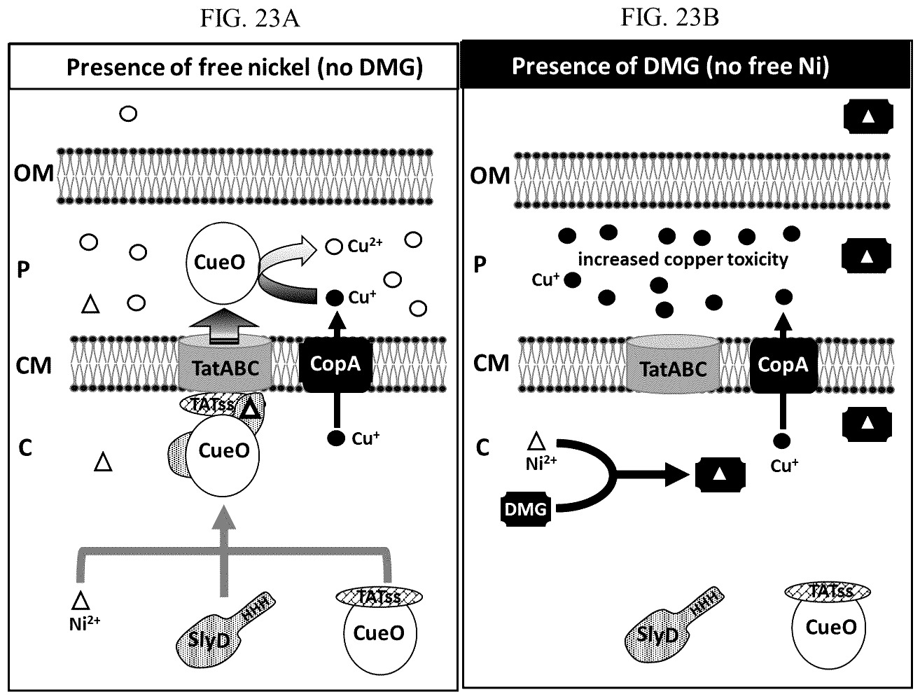

Without wishing to be bound by theory, FIGS. 23A and 23B provide a proposed mode of action mechanism of DMG in C. jejuni. Referring to FIG. 23A, under normal nickel homeostasis (absence of DMG), nickel ions (Ni²⁺, open triangles) bind to the histidine-rich C-terminus of the SlyD chaperone. Ni-bound SlyD interacts with the twin arginine translocation (TATss) signal sequence of the copper oxidase CueO, enabling translocation of CueO to the periplasm through the twin arginine translocase (TatABC, in grey). Periplasmic CueO oxidizes cuprous copper (Cu⁺, black circles) into the less toxic cupric copper form (Cu²⁺, open circles). Cu⁺ is transported from the cytoplasm to the periplasm by the membrane-bound P-type ATPase CopA. In presence of the Ni-chelator DMG (FIG. 23B), Ni-depleted SlyD is unable to perform its role as a CueO chaperone. CueO is present in the cell but cannot be translocated to the periplasm. CopA-exported Cu⁺ accumulates in the periplasm, thus increasing copper toxicity.

### Compositions Including a Chelator Active Agent

In another aspect, the present disclosure provides a composition including, for example, that includes as an active agent, a chelator as described herein, and a carrier. In some embodiments, the chelator includes DMG.

The active agent is formulated in a composition and then, in accordance with the methods of the disclosure, administered to a subject. In some embodiments, the subject is a vertebrate, particularly a mammal, such as a human patient, companion animal, or domesticated animal. In some embodiments the domesticated animal is domesticated fowl. In some embodiments, the domesticated fowl is a chicken. The composition is formulated in a variety of forms adapted to the chosen route of administration. In some embodiments, the preferred route is oral administration.

Oral administration of the DMG containing composition to a domesticated animal, particularly domesticated fowl, includes a carrier. In some embodiments, the carrier is an aqueous liquid. In some embodiments, the carrier is a food product.

The aqueous liquid carrier may include an elixir. In some embodiments, the elixir may contain 5%-40% by volume ethanol. In some embodiments, the elixir is substantially free of ethanol. In some embodiments, the elixir is essentially free of ethanol. In some embodiments, the elixir is free of ethanol. The elixir may contain additives. The elixir addictive may be a sugar. Examples of sugars include, but are not limited to, glucose (dextrose), sucrose (saccharose), lactose, maltose, galactose, mannose, trehalose, arabinose, dextrin, ribose, xylose, and combinations thereof. The sugar elixir additives may be already formulated as food products such as agave syrup, barley malt syrup, beet sugar, birch syrup, brown sugar, cane sugar, carbo sugar, coconut sugar, confectioner's sugar, corn sugar, corn syrup, date sugar, cane juice, honey, fruit juice, maltodextrin, maple sugar, maple syrup, molasses, powdered sugar, rice malt, sweet sorghum, treacle, sugar cane, or combinations thereof. The elixir additive may be a flavor enhancer. Flavor enhancers include, but are not limited to, aspartame, salt, artificial flavors, natural flavors, artificial sweeteners, or combinations thereof. In some embodiments, the aqueous liquid contains nutrient supplements including, but not limited to, electrolytes, vitamins, minerals, prebiotics, and combinations thereof.

The food product carrier may be any food product suitable for oral consumption by a subject, such as a domesticated animal. The active agent may be incorporated into the animal's diet by incorporating the active agent into the animal's feed. The active agent may be incorporated into crumbles, pellets, and mash food products. The active agent containing food product may also contain one or more of the following: a binder such as gum tragacanth, acacia, corn starch, or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent.

In some embodiments, the carrier includes an additional active agent. In some embodiments, the additional active agent preferably includes an antibacterial compound. In some embodiments, the antibacterial compound includes copper. In some embodiments, the additional active agent may include a metallic ion or an antibacterial compound that produces a metallic ion.

In some embodiments, when the antibacterial compound includes copper, the copper may include copper(I) or copper(II). In some embodiments, including, for example when a composition or compound is intended to be administered to a subject suffering from or susceptible to a Campylobacter jejuni infection, the metallic ion may preferably be Cu²⁺. As described in Example 4, while millimolar levels of DMG are bacteriostatic against C. jejuni, the addition of micromolar levels of copper(II) renders millimolar levels of DMG bactericidal towards C. jejuni. This effect was unexpected and was serendipitously discovered. While testing the ability of nickel to inhibit DMG-mediated growth inhibition of C. jejuni, zinc and copper were used as a control. Rather than copper having no effect of the ability of DMG to inhibit the growth of C. jejuni, an increased effect on the ability of DMG to inhibit the growth of C. jejuni was observed.

In some embodiments, the metallic ion is preferably a divalent cation. Exemplary divalent cations include copper(II) (Cu²⁺); cobalt(II) (Co²⁺); manganese(II) (Mn²⁺); zinc(II) (Zn²⁺); and tin(II) (Sn²⁺). In some embodiments, the divalent cation does not include nickel, or, because of their toxicity to subjects, cadmium(II) (Cd²⁺), mercury(II) (Hg²⁺) or lead(II) (Pb²⁺).

In embodiments when the antibacterial compound produces a metallic ion, the compound may preferably be a salt or another compound that produces a metal ion upon being dissolved, for example, in water or another pharmaceutical carrier. Exemplary salts include, CuSO₄, CoCl₂, MnSO₄, and ZnSO₄ and hydrates thereof. In some embodiments one salt is used. In some embodiments, two or more salts are used.

In another aspect, the present disclosure provides a composition including, for example, a pharmaceutical composition, that includes as an active agent, a chelator as described herein, and a pharmaceutically acceptable carrier. In some embodiments, the chelator includes DMG.

The active agent is formulated in a pharmaceutical composition and then, in accordance with the method of the disclosure, administered to a subject. In some embodiments, the subject is a vertebrate, particularly a mammal, such as a human patient, companion animal, or domesticated animal, in a variety of forms adapted to the chosen route of administration. A pharmaceutical composition includes a composition suitable for oral, rectal, vaginal, topical, nasal, ophthalmic, or parenteral (including subcutaneous, intramuscular, intraperitoneal, and intravenous) administration.

The pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In some embodiments, the pharmaceutically acceptable carrier may include at least one compound that is not naturally occurring or a product of nature.

In some embodiments, the active agent may be formulated in combination with one or more additional active agents, such an antibacterial compound. Any known therapeutic agent can be included as an additional active agent. The action of the additional active agent in the combination therapy can be cumulative to the chelator or it can be complementary, for example to manage side effects or other aspects of the patient's medical condition. In one embodiment, the combination therapy includes at least one compound that is not naturally occurring or a product of nature. In some embodiments, the combination therapy includes an antibiotic including, for example, an antibiotic belonging to the β-lactam class; including, for example, penicillin derivatives, cephalosporins, or carbapenems, or combinations thereof; an antibiotic belonging to the macrolide class including, for example, erythromycin, azithromycin, or clarithromycin, or combinations thereof; an antibiotic belonging to the glycopeptide class including, for example, vancomycin; an antibiotic belonging to the fuoroquinolone class including, for example, ciprofloxacin or levofloxacin, or a combination thereof, or an antibiotic belonging to the aminoglycoside class including, for example, gentamycin, neomycin, or streptomycin, or a combination thereof, a metal or salt of a metal, or a combination thereof.

In some embodiments, the additional active agent preferably includes an antibacterial compound. In some embodiments, the antibacterial compound includes copper. In some embodiments, the additional active agent may include a metallic ion or an antibacterial compound that produces a metallic ion.

In some embodiments, when the antibacterial compound includes copper, the copper may include copper(I) or copper(II). In some embodiments, including, for example when a composition or compound is intended to be administered to a subject suffering from or susceptible to a Campylobacter jejuni infection, the metallic ion may preferably be Cu²⁺.

In some embodiments, the metallic ion is preferably a divalent cation. Exemplary divalent cations include copper(II) (Cu²⁺); cobalt(II) (Co²⁺); manganese(II) (Mn²⁺); zinc(II) (Zn²⁺); and tin(II) (Sn²⁺). In some embodiments, the divalent cation does not include nickel, or, because of their toxicity to subjects, cadmium(II) (Cd²⁺), mercury(II) (Hg²⁺) or lead(II) (Pb²⁺).

In embodiments when the antibacterial compound produces a metallic ion, the compound may preferably be a salt or another compound that produces a metal ion upon being dissolved, for example, in water or another pharmaceutical carrier. Exemplary salts include, CuSO₄, CoCl₂, MnSO₄, and ZnSO₄ and hydrates thereof. In some embodiments one salt is used. In some embodiments, two or more salts are used.

The formulations can be conveniently presented in unit dosage form and can be prepared by any of the methods well-known in the art of pharmacy. In some embodiments, a method includes the step of bringing the active agent into association with a pharmaceutical carrier. In general, the formulations are prepared by uniformly and intimately bringing the active compound into association with a liquid carrier, a finely divided solid carrier, or both, and then, if necessary, shaping the product into the desired formulations.

Formulations of the present disclosure suitable for oral administration can be presented as discrete units such as tablets, troches, capsules, lozenges, wafers, or cachets, each containing a predetermined amount of the active agent as a powder or granules, as liposomes, or as a solution or suspension in an aqueous liquor or non-aqueous liquid such as a syrup, an elixir, an emulsion, or a draught. The tablets, troches, pills, capsules, and the like can also contain one or more of the following: a binder such as gum tragacanth, acacia, corn starch, or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent can be incorporated into preparations and devices in formulations that may or may not be designed for sustained release.

Formulations suitable for parenteral administration conveniently include a sterile aqueous preparation of the active agent, or dispersions of sterile powders of the active agent, which are preferably isotonic with the blood of the recipient. Parenteral administration of a chelator (for example, through an intravenous drip) is one form of administration. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectable solutions. The likelihood and/or extent of subsequent microbial contamination can be addressed by using various antimicrobial agents, for example, antibacterial, antiviral, antifungal agents, and combinations thereof including parabens, chlorobutanol, phenol, sorbic acid, thimerosal, and the like. Absorption of the active agents over a prolonged period can be achieved by including agents for delaying, for example, aluminum monostearate and gelatin.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration can be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations. Topical formulations can be provided in the form of a bandage, wherein the formulation is incorporated into a gauze or other structure and brought into contact with the skin.

### Administration

A chelator (including, for example, DMG) as the active agent, can be administered to a subject alone or in a composition that includes the active agent and a carrier. In some embodiments, the carrier is an aqueous liquid. In some embodiments, the carrier is a food product. In some embodiments, the carrier is pharmaceutically acceptable carrier. The active agent is administered to a subject such as a vertebrate, particularly a mammal, such as a human patient, companion animal, or domesticated animal, in an amount effective to produce the desired effect. In some embodiments, the domesticated animal is domesticated fowl. In some embodiments, the domesticated fowl is a chicken. A chelator can be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow-release dosage form.

A formulation or composition including the chelator can be administered as a single dose or in multiple doses. Useful dosages of the active agent can be determined by comparing their in vitro activity and the in vivo activity in animal models. Methods for extrapolation of effective dosages in mice, and other animals, to humans are known in the art.

Dosage levels of the active agent in the compositions of this disclosure can be varied to obtain an amount of the active agent which is effective to achieve the desired therapeutic response for a particular subject, composition, and mode of administration, without being toxic to the subject. The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the chelator, the age, sex, weight, condition, general health, and prior medical history of the subject being treated, and like factors well known in the medical arts.

Dosages and dosing regimens that are suitable for other chelators may be suitable for therapeutic or prophylactic administration of the chelators of the present disclosure (including, for example, DMG). Dosages or dosing regimens in use for other chelators, including, for example, sodium diethyldithiocarbamate (DCC), disulfiram, ethylene diamine tetra acetate (EDTA), and 2,3-dimercaptopropanol (BAL), may serve as guideposts for developing suitable animal and human dosages and dosing regimens.

In an exemplary embodiment, the chelator (for example, DMG) may be administered to a subject in an amount of at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg. In exemplary embodiment, the chelator may be administered to a subject in an amount of up to 40 mg/kg, up to 50 mg/kg, up to 60 mg/kg, up to 70 mg/kg, up to 80 mg/kg, up to 90 mg/kg, up to 100 mg/kg, up to 500 mg/kg, or up to 1000 mg/kg. In an exemplary embodiment, DMG may be administered to a subject orally, intravenously, or intramuscularly.

A physician or veterinarian having ordinary skill in the art can readily determine and prescribe the effective amount of the composition required. For example, the physician could start doses of the chelator employed in the pharmaceutical composition at levels lower than that required in order to achieve the desired therapeutic effect and gradually increase the dosage until the desired effect is achieved.

Administration of a chelator composition may occur before, during, and/or after other treatments. Such combination therapy may involve the administration of a chelator composition during and/or after the use of other antibacterial and/or antifungal agents. The administration a chelator composition may be separated in time from the administration of other antibacterial and/or antifungal agents by hours, days, or even weeks.

### Pathogen Infections

In some embodiments, the chelator (including, for example, DMG) or pharmaceutical composition including the chelator may be administered to a subject suffering from or susceptible to an infection by a pathogen. Additionally or alternatively, a chelator (including, for example, DMG) may be used to treat or reduce the likelihood of an infection by a pathogen.

A pathogenic infection may include, for example, a bacterial infection, a fungal infection, or an infection caused by a non-fungal eukaryotic pathogen. Exemplary pathogens that may be nickel-dependent are provided in Maier and Benoit (Inorganics 2019; 7:80). In some embodiments, a pathogen that includes a nickel-containing enzyme includes a pathogen listed in Table 1A. In some embodiments, a pathogen that includes a nickel-containing enzyme includes a pathogen listed in Table 1B.

Exemplary bacterial infections include but are not limited to an infection with a bacterium that includes a nickel-containing enzyme. In some embodiments, a bacterium that includes a nickel-containing enzyme includes a multi-drug resistant pathogen. In some embodiments, a bacterium that includes a nickel-containing enzyme may include Acinetobacter baumannii, Enterococcus faecium, Escherichia coli, Helicobacter pylori, Haemophilus influenzae, Neisseria gonorrhoeae, Streptococcus pneumoniae, a Campylobacter species, an Enterobacter species, a Klebsiella species (including, for example, K. pneumoniae), a Morganella species, a Proteus species, a Providencia species, a Pseudomonas species (including, for example, Pseudomonas aeruginosa), a Salmonella species (including, for example, S. enterica serovar Typhi and S. enterica serovar Typhimurium), a Serratia species, a Shigella species, or a Staphylococcus species (including, for example, Staphylococcus aureus).

Exemplary fungal infections include but are not limited to fungi that include a nickel-containing enzyme. In some embodiments, a fungus that includes a nickel-containing enzyme may include Cryptococcus neoformans, Cryptococcus gattii, Coccidioides posadasii, Histoplasma capsulatum, or Paracoccidioides brasiliensis.

Exemplary infections caused by a non-fungal eukaryotic pathogen include but are not limited to eukaryotes that include a nickel-containing enzyme. In some embodiments, a eukaryote that include a nickel-containing enzyme may include Pythium insidiosum, Leishmania major, Leishmania donovani, or Trypanosoma cruzi.

Exemplary nickel-containing enzymes include a hydrogenase, a urease, a Glyoxalase I, a acireductone dioxygenase, a superoxide dismutase etc. In some embodiments, the bacteria may further include an accessory protein. An accessory protein involved in Ni-dependent hydrogenase maturation may include, for example, a Hyp protein (for example, HypA, HypB, HypC, HypD, HypE, HypF, or HypG) or a homolog thereof. An accessory protein involved urease activation may include, for example, a Ure protein (for example, UreD, UreF, UreG, or UreH).

In some embodiments, a chelator can also be administered prophylactically, to reduce the likelihood and/or extent of a pathogenic infection, for example a bacterial infection, a fungal infection, or an infection caused by a non-fungal eukaryotic pathogen. Treatment that is prophylactic, for instance, can be initiated before a subject manifests symptoms of a pathogenic infection. An example of a subject that is at particular risk of developing a pathogenic infection is an immunocompromised person. Treatment can be performed before, during, or after the diagnosis or development of symptoms of infection. Treatment initiated after the development of symptoms may result in decreasing the severity of the symptoms of one of the conditions, or completely removing the symptoms. A chelator can be introduced into the subject at any stage of pathogenic infection.

Administration of the chelator composition may be, for example, part of a small- or large-scale public health infection control program. The compound may, for example, be added to animal feed and/or drink as a prophylactic measure for reducing, controlling or eliminating a bacterial and/or fungal infection in a wild or domestic animal population. The compound may, for example, be administered as part of routine or specialized veterinary treatment of a companion or domesticated animal or animal population. It should be understood that administration of the compound may be effective to reduce or eliminate bacterial and/or fungal infection or the symptoms associated therewith; to halt or slow the progression of infection or symptoms within a subject; and/or to control, limit or prevent the spread of infection within a population, or movement of infection to another population.

Administration of a chelator can occur before, during, and/or after other treatments. Such combination therapy can involve the administration of a chelator during and/or after the use of other anti-bacterial, anti-fungal, or anti-non-fungal eukaryotic agents, or a combination thereof. The administration a chelator can be separated in time from the administration of other anti-bacterial, anti-fungal, non-fungal eukaryotic, agents by hours, days, or even weeks.

In some embodiments, the chelator is administered to a subject that has been diagnosed with, or is exhibiting symptoms of, or is at risk of developing, a pathogenic infection, for example a bacterial infection, a fungal infection, or an infection caused by a non-fungal eukaryotic pathogen. In another embodiment, the chelator is administered in a subject or subject population that serves, may serve, or is suspected of serving as an infection reservoir, regardless of the presence of symptoms. Administration can be, for example, part of a small or large scale public health infection control program. The chelator can, for example, be added to animal feed as a prophylactic measure for reducing, controlling, or eliminating pathogenic infection in a wild or domestic animal population. The compound can, for example, be administered as part of routine or specialized veterinary treatment of a companion or domesticated animal or animal population. It should be understood that administration of the chelator can be effective to reduce or eliminate pathogenic infection or the symptoms associated therewith; to halt or slow the progression of infection or symptoms within a subject; and/or to control, limit, or reduce the likelihood of infection spreading within a population, or movement of infection to another population.

### Metal-Related Amyloid-β Peptide Aggregation

In some embodiments, the chelator or pharmaceutical composition including the chelator may be administered to a subject suffering from or susceptible to diseases associated with amyloid-R peptide aggregation. For example, in some embodiments, the subject may be suffering from or susceptible to Alzheimer's Disease (AD), adult Down Syndrome, or some types of cancers.

DMG inhibits in vitro Aβ peptide aggregation (See Example 2 for experimental details.) Briefly, to evaluate Aβ peptide aggregation in vitro, synthetic peptides or recombinant peptides may be used. Recombinant Aβ peptides are expressed in, and purified from, organisms such as E. coli. Synthetic Aβ peptide preparations have been associated with various problems such as presence of impurities in the preparation, incorporation of the D-form of amino-acids (e.g. D-His, D-Met, D-Arg) instead of the L-form of amino acids during synthesis. Additionally, Synthetic Aβ peptide preparations are associated with reproducibility issues in terms of quality and yield, to a point that even batch-to-batch variations have been reported (Dobeli et al. Biotechnology (NY) 13, 988-993 (1995); Zagorski et al. Methods Enzymol 309, 189-204 (1999); Finder et al. Journal of molecular biology 396, 9-18 (2010)). The expression and purification of recombinant Aβ peptides also has limitations and issues, including low yield, reduced solubility and presence of oxidized amino-acids (e.g. Met₃₅-sulfoxide)(Finder et al. Journal of molecular biology 396, 9-18 (2010)). One major difference between synthetic and recombinant Aβ peptides that is often overlooked in the literature, is the absence and presence of metals associated with each preparation, respectively. Any protein or peptide showing natural affinity for one (or several) metal(s), as it is the case with Aβ peptides for copper or zinc (Tõugu et al. Metallomics 3, 250-261 (2011)), will likely encounter and bind to the metals within the host (E. coli or other hosts). Hence, recombinant Aβ peptides are likely to be already associated with metals upon purification, in contrast to synthetic peptides. Given that both Cu and Zn enhance Aβ peptide aggregation, Cu and/or Zn-containing recombinant Aβ peptides may be “naturally” more prone to aggregation than their synthetic counterparts. This could account for differences reported in a study by Finder and coworkers, who found that recombinant Aβ₄₂ peptides (likely metal-bound) aggregated faster and were more neurotoxic than synthetic Aβ₄₂ peptides (likely metal-depleted) (Finder et al. Journal of molecular biology 396, 9-18 (2010)).

A commercial recombinant Aβ₄₀ peptide preparation was subjected to ICP-MS metal analysis. Results unambiguously showed the presence of various metals, including Al, Cu, Mn, Zn, Se and Ni, the two latter elements being by far the most abundant (ppm range). Additional metal analysis of other components of the commercial kit revealed the presence of Al, Cu, Fe, Mn, and Ni, but no Se. Furthermore, components-associated Ni levels were negligible compared to the peptide-associated Ni levels. Metallic ions, more especially Cu(II) and Zn(II), enhance in vitro aggregation of both Aβ₄₀ and Aβ₄₂ peptides (Ricchelli et al. Cellular and Molecular Life Sciences CMLS 62, 1724-1733 (2005); Bush et al. Science 265, 1464-1467 (1994); Chen et al. Inorganic chemistry 48, 5801-5809 (2009); Huang et al. J Biol Chem 272, 26464-26470 (1997); Atwood et al. J Biol Chem 273, 12817-12826 (1998)). Based on these results Ni(II) is likely an Aβ peptide-aggregating metal.

A thioflavin-based (ThT) assay showed that Ni(II) enhances Aβ₄₀ aggregation, whereas DMG-mediated Ni-chelation inhibits it Aβ₄₀ aggregation. Moreover, Ni(II) was found to be more efficient than Cu(II), and less efficient than Zn(II), at promoting Aβ₄₀ aggregation, under the conditions tested in our study. Since various parameters (such as pH and temperature) have been previously shown to have an effect on metal-induced aggregation (Atwood et al. J Biol Chem 273, 12817-12826 (1998); Mantyh et al. Journal of neurochemistry 61, 1171-1174 (1993)), the effect of pH on Ni-dependent aggregation was tested. Three buffers with similar salt content (192 mM NaCl) but various pHs (6.5, 7.5, or 8.5) were used. Acidic pH (6.5) conditions increased Ni-induced aggregation compared to the control pH (7.5), whereas Ni-induced aggregation was abolished at pH 8.5. The increased Ni-induced aggregation at acidic pH, is in agreement with previous published data from Atwood et al., who reported an increase of Aβ₁₋₄₀ peptide aggregation in presence of 1 μM Ni at pH 6.6, compared to pH 7.4 (Atwood et al. J Biol Chem 273, 12817-12826 (1998)). Likewise, the same study correlated acidic pH (6.6) with enhanced aggregation, in presence of either Cu or Zn (both at 20 μM). In the present disclosure, Zn-induced aggregation was slightly higher at pH 7.5 compared to 6.5, and significantly faster compared to pH 8.5. The effect of pH on Cu-induced aggregation was negligible, but it is worth noting that the effect of Cu was very limited throughout the ThT-based assays. The effect of temperature on Ni-dependent Aβ₁₋₄₀ peptide aggregation was not tested with the fluorescence-based method, as all assays were carried out at 37° C. However, Ni-Aβ₄₀ peptide binding was also observed at 25° C., as shown by ITC. Although results obtained with both methods cannot be directly compared, Ni binding to Aβ40 peptides happens both at 25° C. and 37° C.

Since aggregation in presence of a particular metal (e.g., nickel) suggests initial metal-peptide binding, the likelihood of Ni binding to Aβ₄₀ peptide was investigated by using ITC. The calorimetry-based method has been successfully used in the past to study Zn binding to Aβ₄₀ peptide, both at low (10 μM) and high (70 μM) concentrations (Talmard et al. Biochemistry 46, 13658-13666 (2007)). Only the effect of Ni on low Aβ₄₀ peptide concentration was examined, with a starting concentration of Aβ₄₀ peptide in the sample cell at 20 μM. After Ni was injected via 20 consecutive injections, every 5 min (5 μM increments), a heat profile characteristic of independent metal-binding was observed. Although the apparent K_{d} (4.2 μM) is similar to that reported for Zn (Talmard et al. Biochemistry 46, 13658-13666 (2007)), the apparent stoichiometry (0.7 mole of Ni per mole of Aβ₄₀) is significantly lower than that previously reported by Drochioiu and colleagues. Drochioiu found that synthetic Aβ₄₀ peptide displays high affinity toward nickel ions with up to three Ni²⁺ ions bound per Aβ₄₀ peptide (Drochioiu et al. Biophys Chem 144, 9-20 (2009)). However, the discrepancy between the results may be due to the nature of Aβ₄₀ peptide used, and the type of analytical method used to analyze Ni-Aβ₄₀ peptide. In the present disclosure, purified recombinant Aβ₄₀ peptide and ITC were used. In contrast, Drochioiu et al. used synthetic Aβ₄₀ peptide, electrospray ion trap mass spectrometry (ESI-MS), and circular dichroism (CD). Aβ₄₀ peptide can bind nickel with high affinity. Furthermore, results confirm that DMG inhibits Aβ₄₀ peptide aggregation through Ni chelation (not direct contact with the peptide), since titration of the peptide with DMG did not induce any peptide conformational change, as observed with ITC.

Given the presence of Cu²⁺, Ni²⁺, Zn₂₊ in recombinant Aβ₄₀ peptide, combined to their respective effect on Aβ₄₀ peptide aggregation, metal chelation therapy towards AD constitutes a valid approach. However, the risk of chelation therapy is that removal of essential metal ions will lead to serious adverse effects (for instance, iron-deficiency anemia) as pointed out by other researchers (Bush et al. Neurotherapeutics 5, 421-432 (2008)). Hence it is preferable to use chelators with select affinity towards non-essential metals: the Ni-specific chelator DMG is therefore a good candidate. Mammalian hosts do not contain known Ni-dependent enzymes, which makes Ni-chelation therapy an attractive approach (Maier et al. Inorganics 7, 80 (2019)). On the other hand, most bacteria, including pathogenic ones, require nickel as cofactor for one or several enzymes, such as [Ni—Fe]hydrogenase(s) (Benoit et al. Microbiology and Molecular Biology Reviews 84, (2020)) or urease (Maier et al. Inorganics 7, 80 (2019)). Thus, DMG-mediated inhibition of these enzymes, as demonstrated with Salmonella Typhimurium hydrogenases or Klebsiella pneumoniae urease, leads to bacterial growth inhibition, both in the mouse and in the wax moth animal models as described in Example 1. In the present study, we showed that DMG can drastically reduce, and even abolish Aβ₄₀ peptide aggregation. The inhibitory effect was observed in absence of supplemental metal, as well as in presence of copper, nickel or even zinc (albeit with lower efficacy).

Reinhardt and colleagues reported beneficial effects of the nickel chelator disulfiram on AD hallmarks, including inhibitory effects on Aβ₄₂ peptide aggregation (Reinhardt et al. Scientific reports 8, 1-13 (2018)). However, this study was not aimed at establishing any link between Ni and AD. The authors found that disulfiram increased synthesis of the metalloproteinase α-secretase, resulting in secretion of the neuroprotective APP cleavage product sAPPα and thus preventing formation of the amyloidogenic βA peptides (Reinhardt et al. Scientific reports 8, 1-13 (2018)). The concentration of disulfiram shown to have inhibitory effects on peptide aggregation was significantly lower compared to DMG concentrations used in our study, however the disulfiram drug is highly toxic, even at low doses, with concentrations higher than 5 μM inducing cytotoxicity (Reinhardt et al. Scientific reports 8, 1-13 (2018)). This finding correlates with previous studies linking disulfiram with negative outcomes, such as elevated nickel levels in rat brains (Baselt et al. Res Commun Chem Pathol Pharmacol 38, 113-124 (1982)), elevated nickel levels in body fluids of patients with chronic alcoholism (Baselt et al. Res Commun Chem Pathol Pharmacol 38, 113-124 (1982); Hopfer et al. Res Commun Chem Pathol Pharmacol 55, 101-109 (1987)), as well as hepatotoxicity in humans (Kaaber et al. Contact Dermatitis 5, 221-228 (1979); Kaaber et al. Derm Beruf Umwelt 35, 209-211 (1987)).

DMG may inhibit Ni-requiring microorganisms. There may be a link between pathogens and AD, known as the “infection hypothesis of AD” (Itzhaki et al. J Alzheimers Dis 51, 979-984 (2016); Fulop et al. Front Aging Neurosci 10, 224 (2018); Honjo et al. Alzheimer's & Dementia 5, 348-360 (2009)). The list of pathogens potentially linked to AD includes viral, fungal and bacterial species. Among bacteria directly or indirectly associated with AD, one can find Helicobacter pylori (Malaguarnera et al. European Journal of Internal Medicine 15, 381-386 (2004); Kountouras et al. Cognitive and behavioral neurology 23, 199-204 (2010)), E. coli (Zhan et al. Neurology 87, 2324-2332 (2016)) and Salmonella Typhimurium (Kumar et al. Sci Transl Med 8, 340ra372 (2016)), all of which require Ni as cofactor for one or several enzymes (Maier et al. Inorganics 7, 80 (2019)). In the case of H. pylori, another protein is relevant to the pathogen/AD link. The gastric pathogen produces abundant amounts (2% of total protein) of a small histidine-rich protein (Hpn) that has been shown to develop amyloid-like fibrils in vitro (Ge et al. Biochimica et Biophysica Acta (BBA)-Molecular Cell Research 1813, 1422-1427 (2011)). The continuous production of Hpn by the bacterium during decades of chronic gastric infection could result in leakage of the protein, first into the bloodstream and eventually into the brain, potentially triggering AD, as hypothesized by Ge and Sun (Ge et al. Medical hypotheses 77, 788-790 (2011)). More generally, an antimicrobial role for Aβ peptides (as part of the brain's ancient immune system) has been proposed, as part of a “new amyloidogenesis model” (Moir et al. Alzheimer's & Dementia 14, 1602-1614 (2018)). The model is based on findings by Kumar and colleagues, who reported (Kumar et al. Sci Transl Med 8, 340ra372 (2016)) that bacterial infection of the brains of transgenic mice result in accelerated Aβ plaque deposition, closely colocalizing with the invading bacteria (in this case, Salmonella) (Kumar et al. Sci Transl Med 8, 340ra372 (2016)).

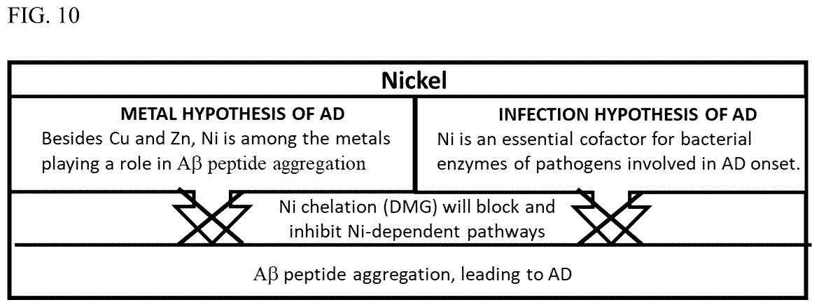

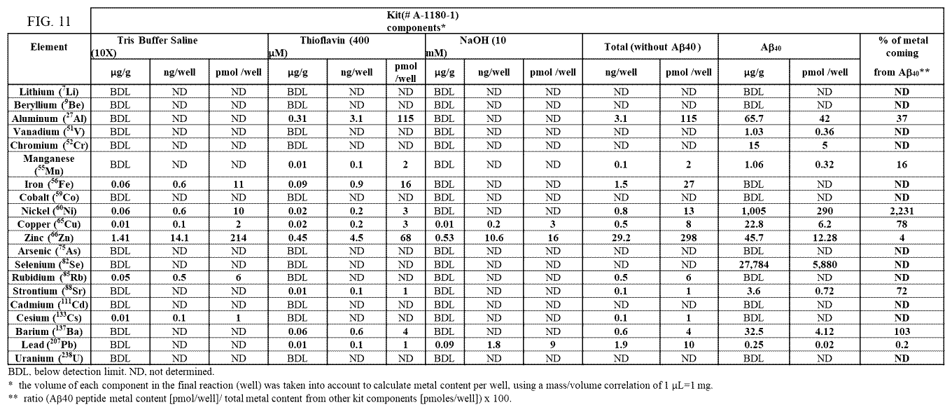

Without wishing to be bound by theory, Ni may affect the onset and the progression of AD through two different mechanisms, as depicted in our proposed model (FIG. 10). The first mechanism involves the binding of Ni²⁺ to Aβ peptides (Aβ₄₀ peptide, possibly Aβ₄₂ peptide), eventually leading to aggregation, plaque formation and AD. This mechanism complies with the metal hypothesis of AD. The second mechanism involves the use of Ni²⁺ as a required cofactor for various enzymes (e.g. Ni-glyoxalase, Ni-superoxide dismutase, Ni-acireductone dioxygenase, [NiFe]hydrogenases and urease, see (Maier et al. Inorganics 7, 80 (2019))) of pathogens previously shown to play a role in Aβ peptide aggregation. Alternatively, some of these pathogens might contribute to AD independently of Aβ peptide plaque formation. Both scenarios would fit the “infectious hypothesis of AD”. Whether one Ni-dependent mechanism is preferred over the other, or both actively contribute to the onset and/or the progression of AD, DMG-mediated Ni-chelation strategy is at the intersection of both (the metal and the infectious) hypotheses. Thus, it is likely to interfere and disrupt both mechanisms, eventually slowing down or stopping the progression of AD.

In some embodiments, a chelator can be administered prophylactically, to reduce the likelihood and/or extent of amyloid-β peptide aggregation. Treatment that is prophylactic, for instance, can be initiated before a subject manifests symptoms of amyloid-β peptide aggregation and/or dementia. An example of a subject that is at particular risk of developing an amyloid-β peptide aggregation is a person with a family history of Alzheimer's Disease or the presence of a genetic mutation associated with Alzheimer's Disease. Treatment can be performed before, during, or after the diagnosis or development of symptoms of dementia. Treatment initiated after the development of symptoms may result in decreasing the severity of the symptoms of one of the conditions, or completely removing the symptoms. A chelator can be introduced into the subject at any stage of dementia.

Administration of a chelator can occur before, during, and/or after other treatments. Such combination therapy can involve the administration of a chelator during and/or after the use of other anti-dementia drugs, anti-Alzheimer's drugs, and/or drugs that reduce the likelihood and/or extent of amyloid-β peptide aggregation. The administration a chelator can be separated in time from the administration of other agents by hours, days, or even weeks.

In some embodiments, the chelator is administered to a subject that has been diagnosed with, or is exhibiting symptoms of, or is at risk of developing amyloid-β peptide aggregation. It should be understood that administration of the chelator can be effective to reduce or eliminate amyloid-β peptide aggregation or the symptoms associated therewith and/or to halt or slow the progression of amyloid-β peptide aggregation or symptoms within a subject.

Alteration of a Subject's Microbiome and/or Obesity

In some embodiments, the chelator or pharmaceutical composition including the chelator may be administered to a subject suffering from or susceptible to a nickel allergy. In some embodiments, the subject may be obese. In some embodiments, the chelator or pharmaceutical composition including the chelator may be administered to a subject to alter the balance of bacteria in the subject's microbiome.

Recent studies have isolated nickel-resistance bacteria from the human microbiome (Lusi et al. 2017 New Microbe and New Infect 19: 67-70) and have shown that obese individuals with a nickel allergy have altered metabolisms compared to non-allergic, weight-matched individuals (Watanabe et al. 2018 PLoS ONE 13(8): e0202683). These finding suggest that chelation of nickel in nickel-allergic individuals and/or individuals with nickel-resistance bacteria in their microbiome may provide a therapeutic benefit for those individuals including, for example, by altering the balance of bacteria in their microbiome and/or the individual's metabolism. The human intestinal microbiota (the intestinal microbiome) impacts many areas of human health, while some of the commensal bacteria in the gut use nickel as a component of enzymes. Therefore, alteration of nickel levels via nickel chelation is expected to alter intestinal microbiome content, potentially providing health benefits.

Administration of a chelator can occur before, during, and/or after other treatments. Such combination therapy can involve the administration of a chelator during and/or after the use of other treatments for nickel allergy. In an exemplary embodiment, DMG may be administered with another chelator including, for example, EDTA. The administration of a chelator can be separated in time from the administration of another agent by hours, days, or even weeks.

### Inhibition or Disruption of Biofilms

In some embodiments, the chelator or a composition including the chelator may be used to reduce the likelihood and/or extend of biofilm formation. Treatment of a surface initiated after the development of a biofilm may result in the death of some of the bacterial cells within the biofilm or all of the bacterial cells within the biofilm. Treatment of a surface initiated before the development of a biofilm may result in a reduction of the likelihood of biofilm formation or slowing the growth of a biofilm.

In some embodiments, the biofilm may include a Campylobacter species (including, for example, C. jejuni and/or C. concisus), Helicobacter pylori, a Klebsiella species, (including, for example, Klebsiella pneumoniae), a Proteus species, a Pseudomonas species (including, for example, Pseudomonas aeruginosa), a Salmonella species (including, for example, S. Typhimurium), or a Staphylococcus species (including, for example, Staphylococcus aureus), or a combination thereof. In some embodiments, the biofilm may include catheter-associated bacteria and/or medical tubing-associated bacteria.

In some embodiments, the biofilm may be present on a medical device including, for example, a catheter, medical tubing, and/or an indwelling device. Additional specific examples of medical devices on which a biofilm may be present include but are not limited to a nasogastric tube, a urinary catheter, a central venous catheter, an umbilical line, an endotracheal tube, a contact lens, a heart valve, a prosthetic device, etc. In some embodiments, a medical device and/or a surface of a medical device may be pre-treated to reduce the likelihood of biofilm formation or to slow the growth of a biofilm.

In some embodiments, treatment of a surface includes application and/or deposition of the chelator to the surface; ionic binding of the chelator to the surface; or incorporation of the chelator to a polymeric matrix bound to the surface.

Bacterial biofilms often prevent or reduce clearance by antibiotics and enable persistent infections in patients. Yonezawa et al. 2013 PLoS One 8:e73301; Davies D. 2003 Nature Reviews Drug discovery 2:114; Vuotto et al. 2017 Journal of Applied Microbiology 123:1003-1018; Stewart 2015 Microbiology Spectrum 3(3). Data described in Example 3 indicates that DMG has biofilm-inhibitory properties against multidrug resistant Salmonella and Klebsiella strains, as well as against the gastric pathogen Helicobacter pylori.

In some embodiments, a composition including the chelator may include an additional active agent, such an antibacterial compound. In some embodiments the surface being treated with the chelator or a composition including the chelator may preferably include an additional active agent preferably includes an antibacterial compound.

In some embodiments, the additional active agent preferably includes an antibacterial compound. In some embodiments, the antibacterial compound includes copper. In some embodiments, the additional active agent may include a metallic ion or an antibacterial compound that produces a metallic ion.

In some embodiments, when the antibacterial compound includes copper, the copper may include copper(I) or copper(II). In some embodiments, including, for example when the chelator or a composition including the chelator is being used to reduce the likelihood and/or extent of biofilm formation including Campylobacter jejuni or to disrupt a biofilm including C. jejuni, the metallic ion may preferably be Cu²⁺. As described in Example 4, while millimolar levels of DMG are bacteriostatic against C. jejuni, the addition of micromolar levels of copper (II) renders millimolar levels of DMG bactericidal towards C. jejuni.

### Exemplary Aspects

The invention is defined in the claims. However, below there is provided a non-exhaustive listing of non-limiting exemplary aspects. Any one or more of the features of these aspects may be combined with any one or more features of another example, embodiment, or aspect described herein.

Aspect 1. Aspect 1 is composition comprising: a chelator wherein the chelator comprises soluble DMG; and a carrier.
Aspect 2. Aspect 2 is the composition of Aspect 1, wherein the carrier is a pharmaceutically acceptable carrier.
Aspect 3. Aspect 3 is the composition of Aspect 1-2, wherein the carrier comprises an aqueous liquid elixir.
Aspect 4. Aspect 4 is the composition of Aspects 1-3, wherein the aqueous liquid elixir comprises a sugar.
Aspect 5. Aspect 5 is the composition of Aspects 1-4, wherein the carrier comprises a food product.
Aspect 6. Aspect 6 is the composition of Aspects 1-5, further comprising an additional active agent.
Aspect 7. Aspect 6 is the composition of Aspects 1-6, wherein the additional active agent comprises a metallic ion or a compound that produces a metallic ion.
Aspect 8. Aspect 8 is the composition of Aspects 1-7, wherein the additional active agent comprises a divalent cation.
Aspect 9. Aspect 9 is a method for administering the composition of Aspects 1-8 to a subject.
Aspect 10. Aspect 10 is the method of Aspect 9, wherein the subject comprises a human or an animal.
Aspect 11. Aspect 11 is the method of Aspects 9-10, wherein the animal comprises a chicken.
Aspect 12. Aspect 12 is the method of Aspects 9-11, wherein the composition further comprises copper.
Aspect 13. Aspect 13 is the method of Aspects 9-12, wherein the subject is infected with a pathogen or susceptible to infection by a pathogen.
Aspect 14. Aspect 14 is the method of Aspects 9-13, wherein the pathogen comprises a nickel-containing enzyme, a fungus that comprises a nickel-containing enzyme, or a non-fungal eukaryotic pathogen that comprises a nickel-containing enzyme Aspect 15. Aspect 15 is the method of Aspects 9-14, wherein the pathogen comprises a multi-drug resistant pathogen.
Aspect 16. Aspect 16 is the method of Aspects 9-15, wherein the pathogen comprises
Acinetobacter baumannii, Enterococcus faecium, Escherichia coli, Helicobacter pylori, Haemophilus influenzae, Neisseria gonorrhoeae, Streptococcus pneumoniae, a Campylobacter species, an Enterobacter species, a Klebsiella species, a Morganella species, a Proteus species, a Providencia species, a Pseudomonas species, a Salmonella species, a Serratia species, a Shigella species, or a Staphylococcus species, or a combination thereof,
Cryptococcus neoformans, Cryptococcus gattii, Coccidioides posadasii, Histoplasma capsulatum, or Paracoccidioides brasiliensis, or a combination thereof; or
Pythium insidiosum, Leishmania major, Leishmania donovani, or Trypanosoma cruzi, or a combination thereof.
   Aspect 17. Aspect 17 is the method of Aspects 9-16, wherein the subject is suffering from or susceptible to a disease associated with amyloid-β peptide aggregation.
   Aspect 18. Aspect 18 is the method of Aspects 9-17, wherein the disease is Alzheimer's, Down Syndrome, or both.
   Aspect 19. Aspect 19 is the method of Aspects 9-18, wherein the method comprises treating or preventing β-Amyloid peptide aggregation in a subject, the method comprising administering dimethylglyoxime (DMG) to the subject.
   Aspect 20. Aspect 20 is the method of Aspects 9-19, wherein the DMG comprises soluble DMG.
   Aspect 21. Aspect 21 is the method of Aspects 9-20, wherein the soluble DMG comprises disodium salt DMG, or disodium salt octahydrate DMG, or both.
   Aspect 22. The method of Aspects 9-21, wherein the subject is suffering from or susceptible to Alzheimer's Disease or Down Syndrome or both.
   Aspect 23. The method of Aspects 9-22, wherein DMG is administered in combination with another anti-dementia therapy.
   Aspect 24. The method of Aspects 9-23, wherein DMG is administered orally or intravenously.
   Aspect 25. The method of Aspects 9-24, wherein the method comprises administering DMG orally, and wherein the DMG is presented as a capsule.
   Aspect 26. The method of Aspects 9-25, wherein the DMG comprises soluble DMG.
   Aspect 27. Aspect 27 is the method of Aspects 9-26, wherein the method comprises disrupting a biofilm or preventing biofilm formation, the method comprising treating a surface with dimethylglyoxime (DMG).
   Aspect 28. Aspect 28 is the method of Aspects 9-27, wherein the biofilm comprises a Campylobacter species, Helicobacter pylori, a Klebsiella species, a Proteus species, a Pseudomonas species, a Salmonella species, or a Staphylococcus species, or a combination thereof.

The present disclosure is illustrated by the following examples. It is to be understood that the particular examples, materials, amounts, and procedures are to be interpreted broadly in accordance with the scope and spirit of the invention as set forth herein.

### EXAMPLES

### Example 1—Nickel Chelation Therapy as an Approach to Combat Multi-Drug Resistant Enteric Pathogens

### Results and Discussion

### the Nickel-Specific Chelator DMG Inhibits Growth of Various Enterobacteriaceae.

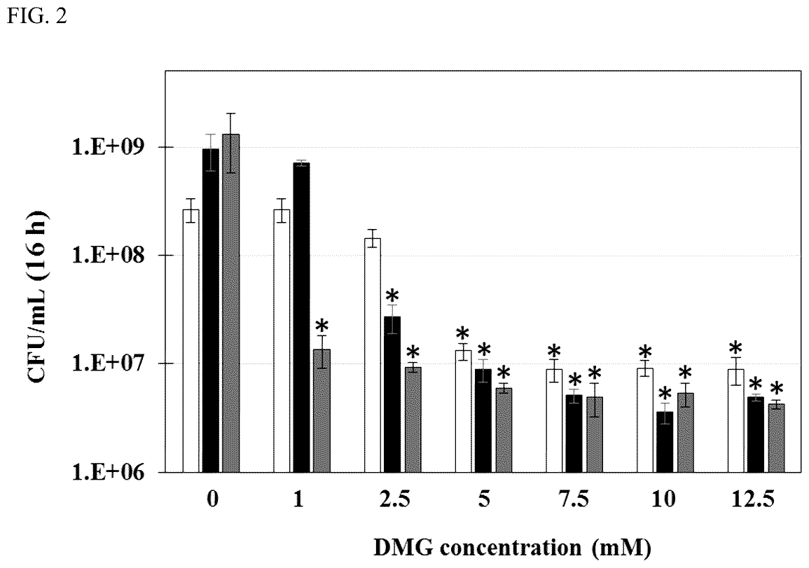

The inhibitory effect of DMG on bacterial growth was tested on three strains: MDR K. pneumoniae (ATCC BAA2472), MDR S. Typhimurium (ATCC 700408), and S. Typhimurium (ATCC 14028), the last of which is a mouse colonizing strain (Gunn et al. 2000 Infect. Immun. 68:6139-6146). Cells were inoculated at a starting OD₆₀₀ of 0.005 (approximately 5×10⁶ CFU/mL) and grown for 24 hours at 37° C. under aerobic conditions with vigorous shaking in presence of defined concentrations of DMG, as indicated, and the growth yield (CFU/mL) was determined after serial dilutions and plating (FIG. 2). While there was no measurable effect of DMG at 2.5 mM, 5 mM, or 7.5 mM on bacterial growth, more than 99.99% growth inhibition was achieved in presence of 10 mM chelator for all three bacterial strains tested. Since approximately 10⁶ cells per mL were still detected, these results suggest DMG has a bacteriostatic effect on the growth of these Enterobacteriaceae. Thus, millimolar concentrations of DMG can inhibit in vitro growth of various Enterobacteriaceae, including MDR strains of K. pneumoniae and S. Typhimurium.

### Sublethal DMG Levels Abolish S. Typhimurium Hydrogenase Activity.

To study the effect of Ni-chelation on hydrogenase activity in S. Typhimurium, cells from strain 14028 were grown for six hours on blood-based media containing increasing concentrations of DMG, under H₂-enriched microaerobic atmosphere; these conditions (blood medium and H₂) have been previously shown to be favorable for the expression of all three S. Typhimurium respiratory hydrogenases (Maier et al. 2004 Infect Immun 72:6294-6299). Hydrogenase assays were carried out on whole cells using an amperometric method, as previously described (Maier et al. 2004 Infect Immun 72:6294-6299; Lamichhane-Khadka et al. 2015 Infect Immun 83:311-316). While addition of 0.1 mM DMG to the medium had no effect on the (combined) H₂-uptake hydrogenase activity, supplementation of the growth medium with either 0.5 mM, 1 mM, or 5 mM DMG significantly decreased hydrogenase activity, and addition of 10 mM DMG abolished hydrogenase activity (Table 2). The addition of 50 μM NiCl₂ to a medium containing 1 mM DMG restored some hydrogenase activity (50% increase compared to the 1 mM DMG medium). Taken together, these results indicate that DMG inhibits hydrogenase activity in S. Typhimurium, probably through Ni-chelation. H₂-uptake hydrogenase activity is required for colonization in a murine model. Hence, the inhibitory effect of DMG on hydrogenase activity observed herein suggests the Ni-chelator could inhibit S. Typhimurium growth in animals.

Sublethal DMG Levels Inhibit MDR Klebsiella pneumoniae Urease.

To study the effect of Ni-chelation on urease activity in MDR K. pneumoniae BAA-2472, cells were grown overnight in LB broth supplemented with sublethal concentrations of DMG, cells were harvested, broken, and urease assays were performed on cell-free extracts (Table 3). Supplementation of the growth medium with 1 mM or 2 mM DMG significantly decreased urease activity in MDR K. pneumoniae, while addition of 5 mM completely inhibited the urease activity in the pathogenic strain. Thus, similar to hydrogenase inhibition, it appears DMG-mediated Ni chelation can be used to efficiently block urease activity in Klebsiella. This confirms previous results from Nakashige and coworkers, who showed that calprotectin-driven chelation of nickel led to urease inhibition in K. pneumoniae (Nakashige et al. 2017 J. Am. Chem. Soc. 139:8828-8836). Urease is used in nitrogen metabolism by K. pneumoniae; when tested in a competition experiment with the wild-type strain, an isogenic urease mutant failed to colonize mouse intestines (Maroncle et al. 2006 Res Microbiol 157:184-193). Thus, the inhibition of K. pneumoniae urease by DMG, as shown in the present study, is anticipated to have a major (inhibitory) impact on the in vivo colonization of the pathogen.

TABLE 2
Effect of DMG chelation on hydrogenase activity in S. Typhimurium 14028.

| DMG (mM) ^{a} | NiCl₂ (mM) | Hydrogenase activity ^{b} |
|---|---|---|
| 0 | | 14.7 ± 2.8 |
| 0.1 | | 14.0 ± 2.7 |
| 0.5 | | 4.6 ± 1.2 |
| 1 | | 3.9 ± 0.9 |
| 1 | 0.05 | 6.9 ± 1.7 |
| 5 | | 0.8 ± 0.1 |
| 10 | | ND |

^{a} DMG was added to a blood-based medium, and cells were grown for six hours under H₂-enriched microaerobic conditions before being harvested.
^{b} nmoles H₂ oxidized per minute per 10⁹ cells
Values shown are the mean ± standard deviation for 6 independent replicates.
Results beginning with 0.5 mM of DMG are significantly less than without DMG (P < 0.01%, Student's t-test).

TABLE 3
Effect of DMG chelation on urease activity in K. pneumoniae BAA-2472

| DMG (mM)^{a} | Urease activity^{b} |
|---|---|
| 0 | 0.17 ± 0.03 |
| 1 | 0.05 ± 0.01 |
| 2 | 0.03 ± 0.01 |
| 5 | ND^{c} |

^{a}DMG was added to LB broth, cells were grown overnight, and urease assays were performed on cell-free extracts using the phenol-hypochlorite method of Weatherburn et al. 1967 Analytical Chemistry 39:971-974.
^{b}Urease activity is expressed in μmoles of NH₃ produced per minute per mg of total protein.
^{c}ND, not detected (<0.001)
Values shown are the mean ± standard deviation for at least three independent biological replicates, with assays done in triplicate.
Urease activities measured for all DMG-supplemented conditions are significantly lower compared to the no-DMG control (p < 0.01%, Student’s t-test).

### High Levels of DMG are not Toxic for Mice or Wax Moth Larvae.

While in vitro DMG-mediated inhibition of Salmonella and K. pneumoniae strains appear promising, the relatively high (millimolar) concentrations of DMG required to inhibit these pathogens' growth could impede DMG's in vivo use, due to toxicity concerns. A series of experiments were conducted to evaluate the toxicity of DMG on two animal models, Mus musculus (mice) and Galleria mellonella (greater wax moth). Mice (BALB/c) have been used (typhoid fever-mouse model) to study S. Typhimurium virulence (Maier et al. 2014 PLoS One 9:e11018710; Lamichhane-Khadka et al. 2015 Infect Immun. 83:311-316; Gunn et al. 2000 Infect Immun. 68:6139-6146), and wax moth larvae have been proven to be a reliable model for studying virulence of many pathogens, including K. pneumoniae (Shi et al. 2018 BMC Microbiol. 18:94; Esposito et al. 2018. Front. Microbiol. 9:1463; Insua et al. 2013 Infect. Immun. 81:3552-3565) and S. Typhimurium (Scalfaro et al. 2017 FEMS Microbiol. Lett. 364; 43; Kurstak et al. 1968 Can. J. Microbiol. 14:233-237; Bender et al. 2013 PLoS One 8:e73287; Viegas et al. 2013 Appl. Environ. Microbiol. 79:6124-6133). Mice were subjected to the following DMG treatment: two daily doses of 0.2 mL DMG at 50 mM (˜6.1 mg DMG per day), for four consecutive days. These animals displayed no clear toxicity symptoms (for example, no apparent change in health or behavior compared to the no DMG group control). In another experiment, mice received a daily dose of 0.1 mL 40 mM DMG (˜1.2 mg per day) for 4 days, 0.2 mL of 40 mM DMG (˜2.4 mg per day) for four days, and then two days of 0.2 mL of 100 mM DMG (˜6.1 mg per day). Again, these mice displayed no toxicity symptoms over this course of chelator administration, or for the next three days after cessation of chelator administration (mice were then euthanized). Thus, orally delivered DMG does not appear to be toxic to mice (under the conditions described herein). By comparison, EDTA and its derivatives have been shown to be quite toxic to animals (for a review, see Lanigan et al. 2002 Int J Toxicol 21 Suppl 2:95-142). For example, the acute oral LD₅₀ of Disodium EDTA was found to be 400 mg/kg (Brendel et al. 1953 J Am Pharm Assoc Am Pharm Assoc 42:123-124); this corresponds to approximately 8 mg of Na₂-EDTA for mice with an average weight of 20 g. The Ni-chelator disulfiram is even more toxic, with an oral LD₅₀ of disulfiram for mice reported to be as low as 1.013 mg/kg: this corresponds to approximately 20 μg per mouse. Thus, although no formal toxicity study (for example, LD₅₀) was conducted with DMG, these results suggest oral DMG is less toxic in mice than Na₂-EDTA, and far less toxic that disulfiram. The toxicity of DMG was also assessed in wax moth larvae. Injection of 5 μL of increasing concentrations of DMG (ranging from 25 mM to 400 mM) into the rear proleg of G. mellonella was not detrimental to the larvae, since 70% to 100% of larvae in each group (n=10 for each group) were still alive 72 hours after injection.

### Orally-Administered DMG can be Detected in Mouse Livers.

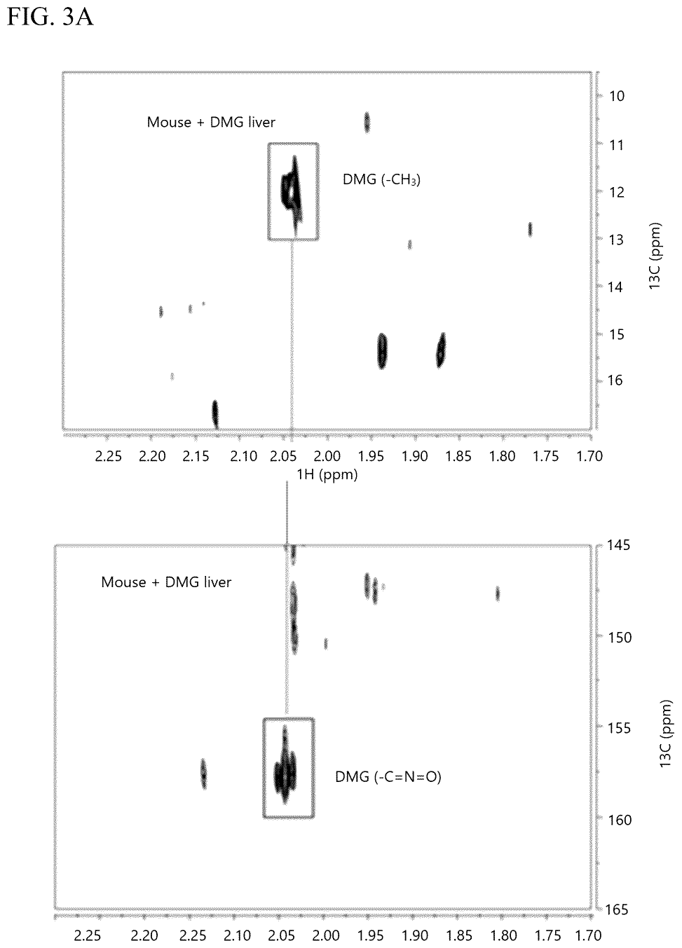

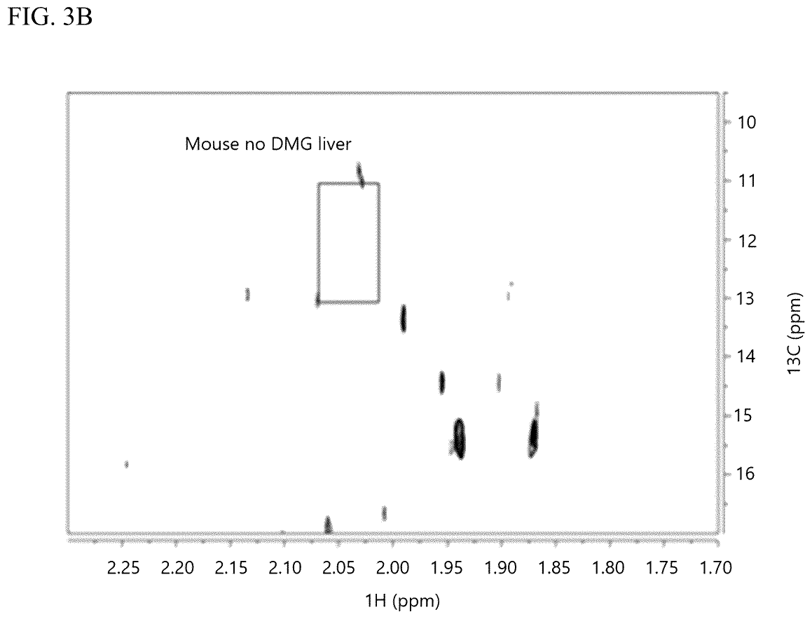

Since water soluble DMG had never been used in animals prior to this study, the intestinal absorption and catabolism of orally delivered DMG in mice was unknown. Therefore, Nuclear Magnetic Resonance (NMR) was used to detect the presence of DMG in liver samples of mice that had been given a daily oral dose (6.1 mg) of aqueous DMG for 2 to 3 days. Although NMR signals from DMG could not be detected in the aqueous supernatant from liver homogenate, diagnostic signals assigned to the methyl carbon (12.0 ppm) and protons (2.04 ppm), and to the oxime carbon (157.8 ppm) were observed in the chloroform extracts of the same supernatant (FIG. 3) (Shaker et al. 2010 Journal of Chemistry 7(S1),S580-S586). In contrast these signals were absent in the chloroform extracts derived from liver samples of the no DMG-control mice (FIG. 3). These results strongly suggest that oral DMG is intestinally absorbed and thus can be detected in the liver indicating that orally-delivered chelator has the potential to inhibit pathogens systemically.

### Oral Delivery of DMG Attenuates Salmonella Virulence in Mice.

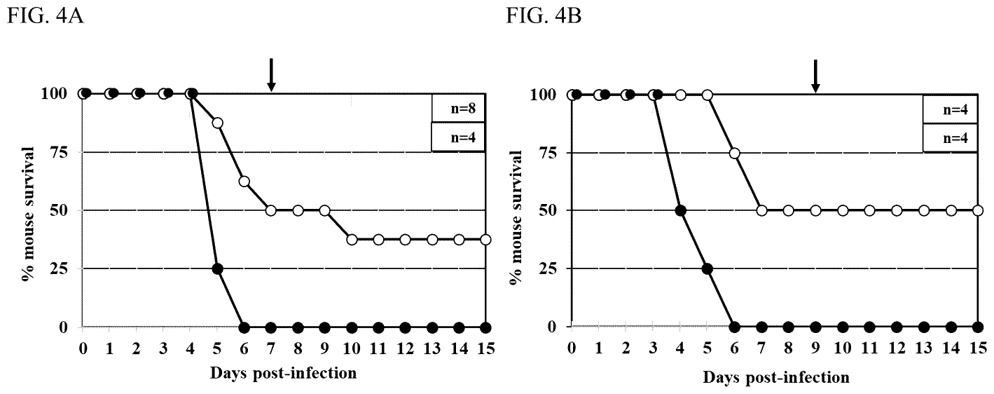

The in vivo efficacy of DMG against S. Typhimurium was assessed in mice, using the mouse-adapted S. Typhimurium strain 14028, as previously described (Gunn et al. 2000 Infect Immun 68:6139-6146). In this typhoid fever-mouse model, the outcome of oral infection with S. Typhimurium is reproducible, typically resulting in a 100% mortality rate within a week (Maier et al. 2004. Infect Immun 72:6294-6299; Maier et al. 2014. PLoS One 9:e11018; Lamichhane-Khadka et al. 2015. Infect Immun 83:311-316). This mortality was confirmed herein: oral infection of mice with 10⁶ bacterial cells (and no DMG) led to 100% mortality within six days, in three independent experiments (see FIG. 4; N=12 total). To determine the efficacy of DMG, a 3-mg dose of the Ni-chelator was orally delivered to mice six hours post-infection and the same treatment (for example, single daily inoculation of 3 mg DMG) was repeated every day for three days, seven days (FIG. 4A) or nine days (FIG. 4B). The three-day DMG treatment postponed for two days the time of death, however it did not change the final outcome (e. g. 100% mortality). By contrast, oral delivery of DMG for seven days (n=8) and nine days (n=4) resulted in 37.5% (FIG. 4A) and 50% mouse survival (FIG. 4B), respectively. Thus, oral administration of nontoxic amounts of DMG to S. Typhimurium-infected mice attenuates bacterial virulence, and even leads to host survival (for up to 50% of animals). These results suggest DMG, and by extension Ni-chelation therapy, can be safely used to eradicate S. Typhimurium in the mouse model host.

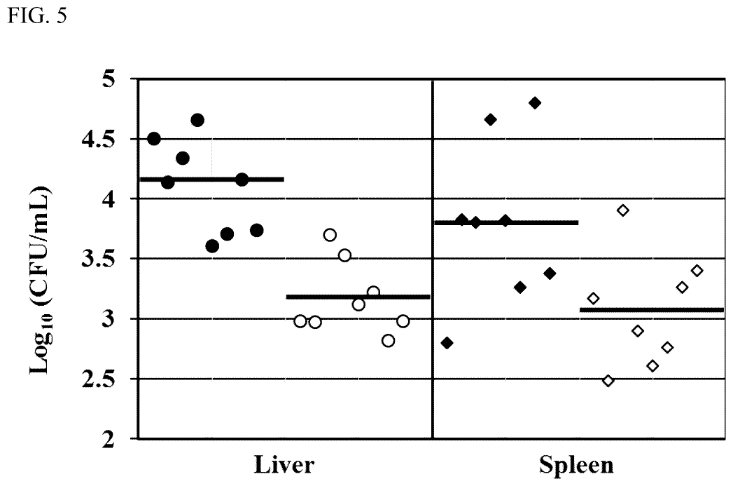

An additional experiment was performed to evaluate the effect of DMG on bacterial loads in key organs (liver and spleen) which are typically colonized in the typhoid fever-mouse model. Two groups (n=8 each) of mice were inoculated with S. Typhimurium 14028; one group was orally given DMG (3 mg) three times (24 hours and 30 minutes before infection, and 24 hours post infection) while the other group did not receive any DMG. The bacterial burden in livers and spleens was determined three days after infection (FIG. 5). Significantly lower bacterial colonization was found in both livers and spleens of DMG-treated animals compared to the non-DMG treated group. These results confirm the capacity of DMG to inhibit S. Typhimurium in vivo, in agreement with the lower death rate observed in DMG-treated animals.

Injection of DMG Reduces Virulence of MDR Enterobacteriaceae in the Galleria mellonella Insect Model.

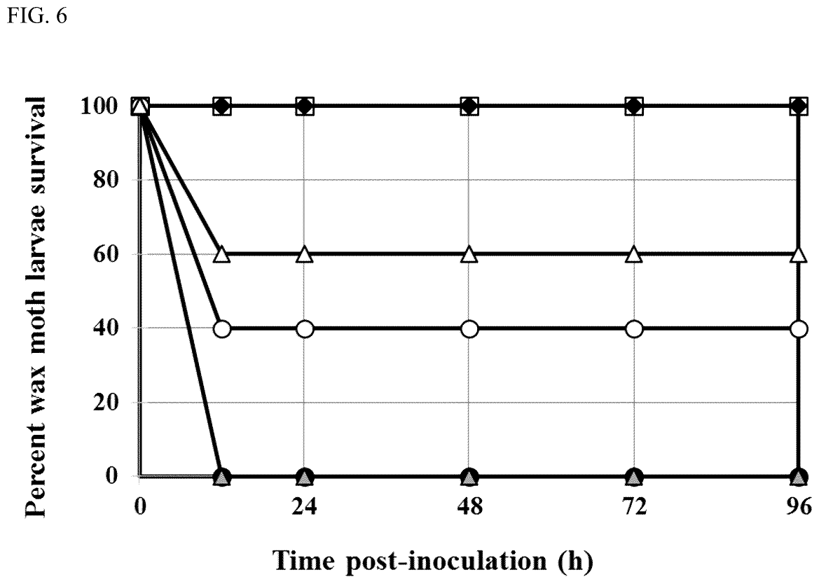

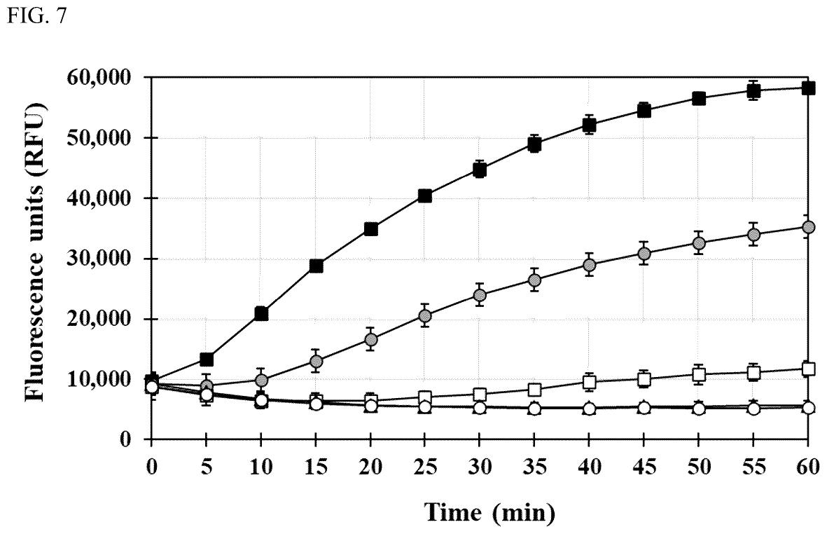

Attempts to establish a reliable K. pneumoniae infection model in mice failed, so an alternate animal model was chosen. As discussed above, the wax moth (G. mellonella) larva model has been already used to study virulence of both K. pneumoniae and S. Typhimurium, and high levels of DMG (5 μL of a 400 mM aqueous solution; approximately 0.61 mg) appear to be innocuous, as determined in this study. Injection of larvae with approximately 10⁶ CFUs of either K. pneumoniae and S. Typhimurium resulted in 100% mortality within 16 hours (FIG. 6). In contrast, when DMG (5 μl of a 250 mM aqueous solution; approx. 0.38 mg) was injected five to ten minutes prior to bacterial challenge, the treatment led to 40% and 60% survival rate for MDR K. pneumoniae and MDR S. Typhimurium, respectively. These results indicate DMG can attenuate both pathogens in vivo in the G. mellonella insect model.

In conclusion, the in vitro and in vivo results presented herein suggest that the growth of Enterobacteriaceae, including that of multi-drug resistant species of K. pneumoniae and S. Typhimurium, can be inhibited by the Ni-chelator DMG. The observed phenotype could be attributable to inhibition of important Ni-enzymes such as hydrogenase and/or urease. Other effects of DMG on cellular metabolism should not be ruled out, however. While full growth inhibition of the pathogens required moderate to elevated levels of DMG, it is worth noting such levels of Ni-chelator appear to be innocuous when tested on two well defined animal models, including, for example, mice and wax moth larvae. These results suggest DMG-mediated chelation should be considered an alternate therapy method, especially when dealing with the ever-growing threat of MDR bacterial species.

### Experimental Procedures

Bacterial strains. The MDR strain of K. pneumoniae subsp. pneumoniae used in this study was ATCC BAA-2472, a New Delhi metallo-beta-lactamase (NDM-1) positive strain, resistant to aminoglycosides, macrolides, fluoroquinolones, and most β-lactams, including ertapenem. S. Typhimurium ATCC 14028 was used for mouse colonization experiments. S. Typhimurium ATCC 700408 is a MDR strain, resistant to ampicillin, chloramphenicol, streptomycin, sulfonamides, tetracycline, among others.

Chemicals. Different batches of DMG were used in this study (all from Sigma-Aldrich, St Louis, MO). For Salmonella hydrogenase inhibition experiments, DMG D1885 (anhydrous) was used. For growth inhibition experiments, DMG D160105 (disodium salt) was used. For all other experiments, including animal studies, DMG 40400 (disodium salt, octahydrate) was used. (See Table 4.)

Growth conditions. All strains were routinely grown on LB agar plates or in LB broth. For DMG-growth inhibition study, S. Typhimurium strains ATCC 14028 and ATCC 700408 were grown in M9 minimal medium (M9 minimal salts, 0.4% glucose, 2 mM MgSO₄, 0.1 mM CaCl₂), and 1 μg/mL thiamine, pH 7.2). K. pneumoniae was grown in “W-U” medium, modified from Bender et al. 1977 129:1001-1009, containing 60 mM K₂HPO₄, 33 mM KH-2PO₄, 0.4 mM MgSO₄, 0.4% Glucose, and 5 mM urea (instead of KNO₃), pH 7.4. Briefly, overnight cultures of S. Typhimurium and K. pneumoniae were harvested, spun at 14,000 rpm for five minutes, washed once with either M9 (S. Typhimurium) or W-U (K pneumoniae) and resuspended in either M9 (S. Typhimurium) or W-U (K. pneumoniae). Cells were inoculated to an OD₆₀₀ of 0.005 in M9 or W-U media, in presence of increasing concentrations of DMG (2.5 mM, 5 mM, 7.5 mM, or 10 mM). Cells were incubated for 24 hours at 37° C. with shaking at 250 rpm, then serially diluted in PBS and 5 μL of each dilution was spotted in triplicate on LB plate. Colony forming units (CFU) were counted after 16 hours at 37° C. Each growth experiment was done at least three times.

Amperometric hydrogenase assays. The hydrogenase activity of S. Typhimurium ATCC strain 14028 was assayed by using an amperometric method, as previously described (Lamichhane-Khadka et al. 2015 Infect Immun 83:311-316). Briefly, cells were grown on blood agar media for six hours under a H₂-enriched microaerobic atmosphere (Maier et al. 2004 Infect Immun 72:6294-6299), supplemented with only DMG (0.1 mM, 0.5 mM, 1 mM, 5 mM, or 10 mM) or 1 mM DMG and 50 μM NiCl₂. Cells were suspended in phosphate-buffered saline (PBS) to a final concentration of 8×10⁸ per mL and added to a sealed amperometric dual-electrode chamber, with constant stirring; 100 μL of H₂-saturated phosphate-buffered saline was added to the chamber and the disappearance of H₂ was recorded over time. Hydrogenase activity is expressed in nmoles of H₂ oxidized per minute per 10⁸ cells.

Urease assays. K. pneumoniae cells were grown overnight at 37° C. in LB (control) or LB supplemented with 1, 2, or 5 mM DMG at 37° C. with shaking at 200 rpm. Cells were pelleted at 6,000 rpm for 30 minutes and washed three times with PBS (pH 7.4). Cells were standardized to an optical density (OD₆₀₀) of 4.5, added to sterile glass beads (0.1 mm diameter, Biospec Products, Bartlesville, OK) at a 100% w/v ratio, and frozen at −80° C. The bead and cell mixture was thawed at room temperature and vortexed at 3,200 rpm for six minutes with one minute intervals on ice. The mixture was pelleted at 14,000 rpm for two minutes and the cell-free supernatant was assayed for urease activity using the phenol-hypochlorite method (Weatherburn et al. 1967. Analytical Chemistry 39:971-974). Protein concentration was determined using the BCA protein kit (Thermo Fisher Pierce, Rockford, IL, USA). Urease activity is expressed as pmoles of NH₃ produced per minute per mg of total protein.

Mouse experiments—Chelator toxicity. A group of eight mice were subjected to the following DMG treatment: two daily doses of 0.2 mL DMG at 50 mM (˜6.1 mg DMG per day) for four consecutive days. These animals displayed no obvious toxicity symptoms. In another experiment, mice received a daily dose of 0.1 mL 40 mM DMG (˜1.2 mg per day) for 4 days, 0.2 mL of 40 mM DMG (˜2.4 mg per day) for four days, and then 0.2 mL of 100 mM DMG (˜6.1 mg per day) for two days. Again, these mice displayed no toxicity symptoms over this course of chelator administration, or for the next three days after cessation of chelator administration (mice were then euthanized).

Mouse experiments—Detection of DMG in liver samples. A group of eight mice was used for this experiment: two mice were given 0.2 mL of 100 mM DMG (˜6.1 mg per day) for two days and then euthanized, and two mice were given the same dose for three days and then euthanized; the remaining four mice were used as no DMG-control. Mice were sacrificed by CO₂ asphyxiation and cervical dislocation. Livers were quickly removed and homogenized in 2 mL sterile deionized water using a tissue homogenizer (“Tissue Tearor” model 985370, Biospec products, Bartlesville, OK, USA). Homogenized liver samples were spun at 16,800×g for six min, and supernatants were collected before being passaged through a 0.45 μm filter unit. Filtered supernatants were subjected to NMR analysis. Since preliminary NMR experiments failed to detect DMG in individual liver samples from DMG-treated mice, these four samples were pooled, concentrated, and also extracted with chloroform for additional NMR analysis. Liver samples from no-DMG treated mice were similarly processed and used as negative controls.

Mouse experiments—Infection Experiments. The in vivo efficacy of DMG against S. Typhimurium was assessed by using the typhoid fever-mouse model, as previously described (Gunn et al. 2000 Infect Immun 68:6139-6146). Female BALB/c mice (Charles River, Boston, MA) were orally inoculated individually with the S. Typhimurium strain ATCC 14028, following previously described methods (Maier et al. 2004 Infect Immun 72:6294-6299; Maier et al. 2014 PLoS One 9:e110187; Lamichhane-Khadka Ret al. 2015 Infect Immun 83:311-316). Briefly, S. Typhimurium cells grown overnight in LB were harvested, washed, and suspended in sterile PBS to a final OD of OD₆₀₀ 0.01 (approximately 10⁷ CFU/mL) and 0.1-mL volumes (10⁶ bacterial cells) were introduced orally into each mouse. A dose of 3 mg of DMG, corresponding to either 0.1 mL of a 100 mM or 0.2 mL of a 50 mM DMG aqueous solution, respectively, was orally given to mice belonging to one group, while the other group of mice (control) was only given sterile H₂O. The DMG treatment was performed six hours post-infection, then (once) every 24 hours post-infection, for three to nine days, as described for each experiment (three independent experiments, with n=4 to n=8 mice for each experiment). The mice were observed twice daily, and morbidity was recorded. In addition, a fourth independent experiment was performed to determine organ (liver and spleen) bacterial burdens. In this experiment, two groups of eight mice each were inoculated with S. Typhimurium 14028. One group was treated for three days with one daily dose of DMG (3 mg), at 24 hours and 0.5 hours before infection, and 24 hours post infection. Infection with S.T. 14028 was done as described above. Mice were euthanized 72 hours after infection. Livers and spleens were removed and homogenized in sterile PBS. Dilutions of the homogenate were plated on bismuth sulfite agar plates (BD DIFCO, Becton Dickinson, Franklin Lakes, NJ), a selective medium for Salmonella species. Colony forming units (CFU) were counted after overnight incubation of the plates at 37° C.

Galleria mellonella (wax moth) experiments. Wax moth larvae were obtained from local pet stores, from two different suppliers: the bug company (Ham Lake, MN; www.ebugco.com) and Timberline (Marion, IL; www.timberlinefresh.com). Larvae were stored in wood shavings at 4° C. in the dark and used within two weeks after purchase. Only larvae weighing 300±50 mg were selected for the experiments. Groups of ten larvae were used for each experiment. The site of injection (last right or left proleg) was disinfected with ethanol 70% (vol/vol) before and after each injection. A 10 μL-Hamilton syringe fitted with a 30.5-gauge needle (Becton Dickinson, Franklin Lakes, NJ) was used to inject 5 μL. After injection, larvae were kept in 9.2-cm Petri dishes at 37° C. in the dark with no food. Larvae were monitored daily for up to days and mortality (as defined by change of pigmentation and absence of response following physical stimulation) was recorded.

Chelator toxicity: the following concentrations of DMG were tested (n=10 for each): 5, 10, 25, 50, 75, 100, 125, 150, 200, 250, 300, 350, 400 mM. Each solution was freshly prepared in 0.8% sterile NaCl, and 5 μL of each solution was injected in the last right proleg. A control with no chelator (0.8% NaCl only) was included in the study. Infection experiment: 5 μL of DMG (250 mM) was injected in the last right proleg of each larva, then approximately 5×10⁵ bacteria (either MDR Kp., or MDR S.T.) were injected in the last left proleg five to ten minutes after DMG inoculation. Bacterial suspensions were prepared as follows: cells were grown overnight in 5 mL of Mueller Hinton broth, harvested, washed once, and resuspended in sterile 0.8% NaCl to a final OD₆₀₀ of 0.1 (approximately 10⁸ CFU/mL, as determined by CFU counts on serially diluted samples). Death rates were compared to those obtained after injection of NaCl only, DMG (250 mM) only, MDR K.p. only, or MDR S. T. only, respectively.

Nuclear magnetic resonance. All data were collected on a Bruker Avance Neo 800 MHz equipped with a 1.7 mM cryoprobe at 25° C. Standard proton spectra (without and with water suppression) and two-dimensional one-bond and multiple-bond proton-carbon correlated spectra (HSQC and HMBC) were collected using Bruker pulse library sequences, zg, zgpr, hsqcetgpsp2.2, and hmbcetgpl3nd, respectively. Data were processed with MNOVA software (Mestrelab Research, Spain). Initial NMR samples were prepared from aqueous liver, spleen, and blood preparations of individual mice, by adding 5 μL of D20 to a 50 μL aliquot from each sample. Since the HSQC spectrum showed no detectable DMG, the two sets of liver samples (with DMG, no DMG) were then separately pooled, lyophilized and 100 μL D20 was added to the residue. Once again, no DMG was detected in the aqueous sample. The two concentrated liver DMG samples were then extracted twice with 1 mL chloroform. The organic phase was separated and dried. 50 μL of CDCl₃ was added to the residue and used for NMR analysis (see FIG. 3). NMR assignments were based on similar compounds described in Shaker et al. (2010 Journal of Chemistry 7(S1), 5580-5586) and were confirmed using authentic aqueous samples of DMG and DMG+NiCl₂ and their chloroform extracts.

TABLE 4

| Reference | Label | | Chemical | FW | Concentration used | | |
|---|---|---|---|---|---|---|---|
| Number | (Manufacturer) | Solubility | formula | (g/mol) | mM | | mg/mL |
| D-1885 | Dimethylglyoxime | Non water soluble | C₄H₈N₂O₂ | 116.12 | 0.1-10 | | 0.011-0.11 |
| | (ALDRICH) | 50 mg/mL in warm ethanol | | | | | |
| D-160105 | Dimethylglyoxime, | High Water solubility | C₄H₆N₂O₂, | 160.1 | 2.5-20 | mM | 0.4-3.2 |
| | disodium salt hydrate, 97% | ~190 g/L or~1.2M (SB) | 2Na | | | | |
| | (SIGMA-ALDRICH) | | | | | | |
| 40400 | Dimethylglyoxime, | High water solubility | C₄H₆N₂O₂, | 304.2 | 50 | mM | 15.2 |
| | disodium salt octahydrate, >97% | ~366 g/L or~1.2M (SB) | 2Na, 8 H₂O | | 100 | mM | 30.4 |
| | | | | | 100 | mM | 30.4 |
| | | | | | 200 | mM | 60.8 |
| | (FLUKA-SIGMA-ALDRICH) | | | | 25-125 | mM | 7.6-38.1 |
| | | | | | 2.5-10 | mM | 0.76-3.0 |
| | | | | | 2.5-20 | mM | 0.76-6.1 |

| Reference | Label | Vol. | | Quantity cmpd | | DMG only | | |
|---|---|---|---|---|---|---|---|---|
| Number | (Manufacturer) | used | | (mg) | | (mg) | | Experiment/Application |
| D-1885 | Dimethylglyoxime | NA | | NA | | NA | | S.T. hydrogenase |
| | (ALDRICH) | | | | | | | inhibition |
| D-160105 | Dimethylglyoxime, | NA | | NA | | NA | | Bacterial (Hp, Kp, ST) |
| | disodium salt hydrate, 97% | | | | | | | growth inhibition |
| | (SIGMA-ALDRICH) | | | | | | | |
| 40400 | Dimethylglyoxime, | 0.2 | mL | 3.04 | mg | 1.16 | mg | Mice/ST inhibition |
| | disodium salt octahydrate, >97% | 0.1 | mL | 3.04 | mg | 1.16 | mg | Mice/ST inhibition |
| | | 0.2 | mL | 6.08 | mg | 2.32 | mg | Mice/NMR |
| | | 0.1 | mL | 6.08 | mg | 2.32 | mg | Mice/NMR |
| | (FLUKA-SIGMA-ALDRICH) | 5 | μL | 38-190 | μg | 14-72 | μg | Wax moth larvae |

| | | | |
|---|---|---|---|
| NA | NA | NA | Kp urease inhibition |
| NA | NA | NA | Growth inhibition (Kp, ST) |

### Example 2—Effect of Metals and DMG-Chelation on Amyloid-β Peptides

Commercial recombinant Aβ₄₀ peptide contains metals, including copper, zinc, and nickel. Most Ab peptide preparations used for in vitro aggregation studies are synthetic (e.g. chemically synthesized) or recombinant peptides (e.g. expressed in prokaryotic or eukaryotic organisms). To determine what type of metals is associated with commercial human recombinant Aβ peptide, a Aβ₄₀ peptide preparation was subjected to a twenty-element ICP-MS analysis (Table 5). Aluminum, copper, manganese and zinc were among the metals found in the Aβ40 peptide preparation, whereas iron was not detected. Surprisingly, the most abundant element associated with the recombinant Aβ₄₀ peptide was selenium, followed by nickel (1 mg of Ni per g of Aβ40 peptide, corresponding to 0.073 moles of Ni per mole of peptide). Taken together, these results suggest that the recombinant Aβ₄₀ peptide used in this study is already metal-bound upon commercialization; metals may be acquired during bacterial expression in the host (E. coli), during the purification process, or both. To our knowledge, it is the first time nickel is found in a commercial recombinant Aβ₄₀ peptide purified preparation, suggesting the peptide (or aggregated peptides) can naturally coordinate the transition metal nickel, in addition to other metals such as aluminum, copper, manganese, selenium and zinc. The metal content of all the other kit components, including TBS, thioflavin and NaOH (used to resuspend the peptide) was also analyzed by ICP-MS (FIG. 11). Aluminum, manganese, iron, nickel copper, and zinc were detected in these components, however no selenium was detected. Besides, the bulk of nickel in the assay (reaction mix) was brought by the peptide (>22-fold more Ni in Aβ₄₀ compared to other kit components, see FIG. 11).

TABLE 5
ICP-MS metal analysis of commercial recombinant Aβ₄₀-peptide.

| | Metal/Aβ40 ratio | |
|---|---|---|
| | μg metal per | mmole metal per |
| Element | g of Aβ₄₀ peptide^{a} | mole of Aβ₄₀ peptide^{b} |
| Lithium (⁷Li) | ND | ND |
| Beryllium (⁹Be) | ND | ND |
| Aluminum (²⁷Al) | 65.7 | 10.5 |
| Vanadium (⁵¹V) | 1.03 | 0.09 |
| Chromium (⁵²Cr) | 15 | 1.25 |
| Manganese (⁵⁵Mn) | 1.06 | 0.08 |
| Iron (⁵⁶Fe) | ND | ND |
| Cobalt (⁵⁹Co) | ND | ND |
| Nickel (⁶⁰Ni) | 1,005 | 72.5 |
| Copper (⁶⁵Cu) | 22.8 | 1.55 |
| Zinc (⁶⁶Zn) | 45.7 | 3.07 |
| Arsenic (⁷⁵As) | ND | ND |
| Selenium (⁸²Se) | 27,784 | 1,470 |
| Rubidium (⁸⁵Rb) | ND | ND |
| Strontium (⁸⁸Sr) | 3.6 | 0.18 |
| Cadmium (¹¹¹Cd) | ND | ND |
| Cesium (¹³³Cs) | ND | ND |
| Barium (¹³⁷Ba) | 32.5 | 1.03 |
| Lead (²⁰⁷Pb) | 0.25 | 0.005 |
| Uranium (²³⁸U) | ND | ND |

ND, not detected (below detection limit)
^{a}background (water) subtracted-values.
^{b}calculated with theoretical molecular mass of 4330 Da.

Addition of nickel enhances Aβ₄₀ peptide aggregation. To determine the effect of nickel on Aβ40 peptide aggregation, a thioflavin-(ThT)-based aggregation kit was used in absence or presence of supplemental Ni(II) (FIG. 7 and Table 6). In absence of supplemental metal, a moderate but steady increase in Aβ₄₀ peptide aggregation was observed (average fluorescence rate of 152 RFU/min, FIG. 7). We hypothesized this might be due to the intrinsic presence of metallic ions, including Cu²⁺, Ni²⁺ and Zn²⁺, as revealed by the ICP-MS metal analysis conducted in the present study (see above). Addition of 10 mM NiSO₄ to the mixture increased the average aggregation rate by 2.5-fold (Table 6), while addition of 100 mM NiSO₄ resulted in a dramatic 5.7-fold increase compared to the no supplemental metal control, suggesting the divalent cation Ni²⁺ can bind to the Aβ₄₀ peptide and enhance its aggregation (FIG. 7 and Table 6). A similar effect was observed when NiCl₂ was used (instead of NiSO₄) as source of Ni²⁺ (data not shown); hence, the nature of the counterion does not appear to play a role in (or interfere with) the observed aggregation. Upon addition of 10 and 100 mM Zn(II), a 5-fold and 14-fold increase in Aβ₄₀ peptide aggregation rate was observed compared to the control, respectively (Table 6), in agreement with previously published studies (Bush et al. Science 265, 1464-1467 (1994); Chen et al. Inorganic chemistry 48, 5801-5809 (2009); Huang et al. J Biol Chem 272, 26464-26470 (1997)); in contrast, addition of 10 or 100 mM CuSO₄ had no significant effect on the aggregation rate (Table 6). This result (lack of aggregation) could be due to the pH used in our study (pH 7.4). Indeed, Cu has been shown to induce Aβ₄₀ peptide aggregation at acidic pH (Atwood et al. J Biol Chem 273, 12817-12826 (1998)), while at neutral pH it is known to promote mostly soluble dimers (Bush et al. J Biol Chem 269, 12152-12158 (1994)).

TABLE 6
Recombinant human Aβ₄₀
aggregation rate as a function of DMG and/or metal.

| | | Relative Aβ₄₀ aggregation rate (% control) | | | |
|---|---|---|---|---|---|
| Supplemental | | Supplemental DMG (μM) | | | |
| metal (μM) | | 0 | 100 | 500 | 1000 |
| A | None (0) | 100 | 33 ± 2 | ND | ND |
| | Ni (10) | 252 ± 8 | 58 ± 11 | ND | ND |
| | Zn (10) | 505 ± 29 | 457 ± 39 | ND | ND |
| | Cu (10) | 103 ± 19 | 54 ± 12 | ND | ND |
| B | None (0) | 100 | 15 ± 14 | <1 | <1 |
| | Ni (100) | 567 ± 61 | 410 ± 91 | <1 | <1 |
| | Zn (100) | 1423 ± 258 | 1468 ± 56 | 833 ± 156 | 328 ± 63 |
| C | None (0) | 100 | 48 ± 8 | 15 ± 5 | 21 ± 10 |
| | Cu (100) | 77 ± 2 | 66 ± 15 | 49 ± 8 | 40 ± 3 |

Addition of DMG inhibits Aβ₄₀ peptide aggregation. Addition of 100 mM of the Ni-specific chelator DMG in absence of supplemented metal severely reduced Aβ₄₀ peptide aggregation, by 40 to 85% depending on experiments (Table 6). Furthermore, addition of 500 mM or 1000 mM DMG led to partial or full inhibition of the aggregation; in the latter case, we measured flat or even decreasing average fluorescence rates (reported as <1% of control, Table 6). Hence this dose-dependent inhibitory effect suggests that (i) DMG is able to pull metals away from the Aβ₄₀ peptide and (ii) the Aβ₄₀ peptide aggregation observed in absence of supplemented metals is likely due to the intrinsic presence of metallic ions (including Ni²+) within the recombinant peptide preparation, since the addition of the chelator leads to inhibition. When increasing amounts of DMG were added to the reaction mixture in presence of 100 mM Ni²⁺, Cu²⁺ or Zn²⁺, results with mixed outcomes were obtained. Complete inhibition was observed in presence of Ni (FIG. 7 and Table 6) and only partial inhibition was seen in presence of Cu or Zn, however Zn was still able to induce Aβ₄₀ peptide aggregation (Table 6). The respective efficacy (or lack thereof) of DMG in presence of Cu, Ni and Zn correlates with the chelator's respective affinity for each metal, as revealed by mass spectrometry analysis of metal-DMG complexes (see below).

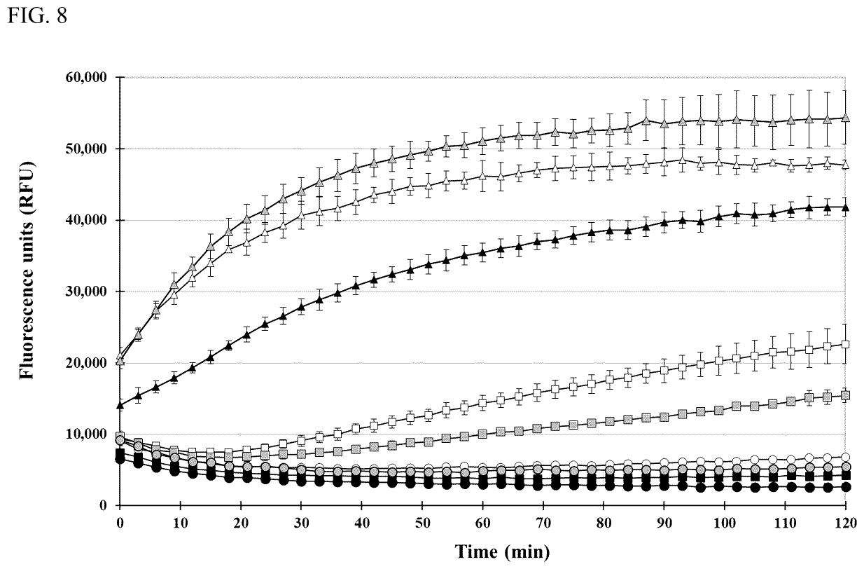

Effect of pH on Aβ₄₀ peptide aggregation in presence of metals or DMG. To study the effect of pH on Aβ₄₀ peptide aggregation (in presence of metals or DMG), additional thioflavin-based aggregation experiments were conducted at pH 6.5, 7.5 or 8.5, with 25 mM Aβ₄₀, in absence or presence of NiSO₄ (25 mM), CuSO₄ (25 mM), ZnSO₄ (10 mM), or DMG (100 mM) (FIG. 8 and data not shown). Overall, aggregation rates at alkaline pH 8.5 (black symbols) were lower compared to pH 7.5 (grey symbols) or pH 6.5 (white symbols). As previously observed, addition of Zn(II) led to the fastest and sharpest increase in fluorescence (triangles) under all pHs tested. Interestingly, Ni(II)-induced aggregation (squares) was faster at pH 6.5, compared to pH 7.5, while it was absent at pH 8.5 (black squares). Ab₄₀ peptide aggregation in presence of Cu(II) or DMG was negligible under all 3 pH conditions tested (data not shown).

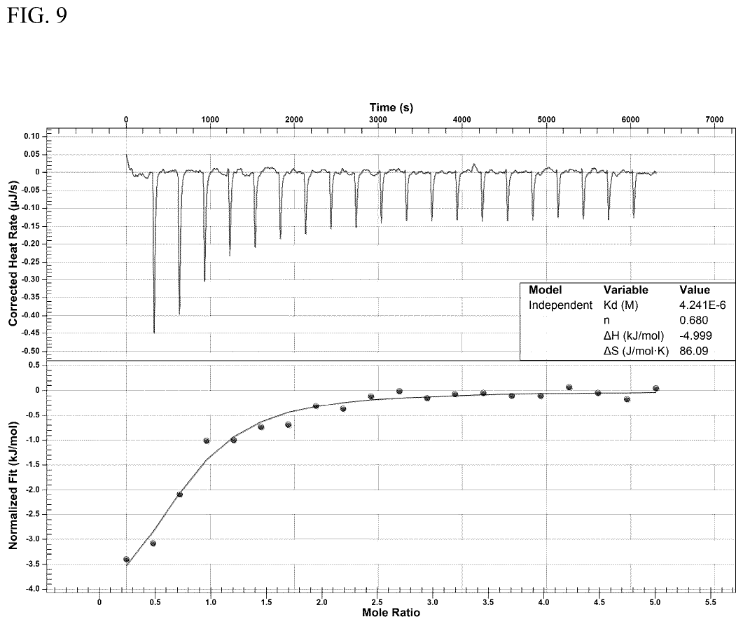

Nickel binding to human recombinant Aβ₄₀ peptide is confirmed by isothermal titration calorimetry. Isothermal titration calorimetry (ITC) has been already used to analyze copper or zinc binding to various Aβ peptides, including Ab₄₀ (Talmard et al. Biochemistry 46, 13658-13666 (2007); Sacco et al. JBIC Journal of Biological Inorganic Chemistry 17, 531-541 (2012); Hatcher et al. The journal of physical chemistry B 112, 8160-8164 (2008)). In the current study, we used ITC to determine whether nickel can bind the Aβ₄₀ peptide. The peptide (same used in ThT-based aggregation assays) was present in the sample cell at a concentration of 20 μM. Twenty injections of NiSO₄ (1 mM solution, 5 μM increments in sample cell) were performed every 5 min under constant stirring (350 rpm) at 25° C., and the heat release was measured (FIG. 9). The heat release profile indicates Ni binding to the peptide (FIG. 9, top Panel). The best fit of Ni titration (FIG. 9, bottom Panel) suggests an apparent stoichiometry of less than 1 mole Ni(II) per mole of Ab₄₀ peptide (˜0.7), in range with previously reported stoichiometry ratios of 1:1 for Cu(II) or Zn(II), and Ab₄₀ (DeToma et al. Chem Soc Rev 41, 608-621 (2012)). The apparent K_{d} value for Ni is approximately 4.2 μM, similar to that previously reported of 7±3 μM for Zn (Talmard et al. Biochemistry 46, 13658-13666 (2007)). Furthermore, the DH (enthalpy) and DS (entropy) were found to be −5 kJ/mol and 86 mol/J/K, suggesting the Ni-Ab₄₀ binding event can be considered both exothermic and spontaneous. Injection of DMG (instead of nickel) into the sample cell containing Ab₄₀ peptide did not induce any significant change, indicating that DMG cannot bind to the peptide (data not shown). Hence, this result suggests the inhibitory effect of DMG on Aβ₄₀ peptide aggregation, as observed with ThT-based assays, is due to nickel chelation, rather than direct DMG-Aβ₄₀ peptide inhibitory interaction.

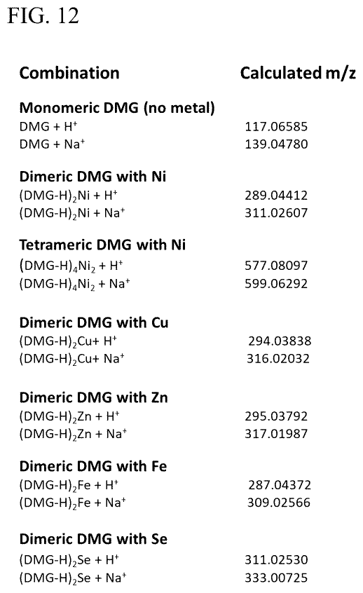

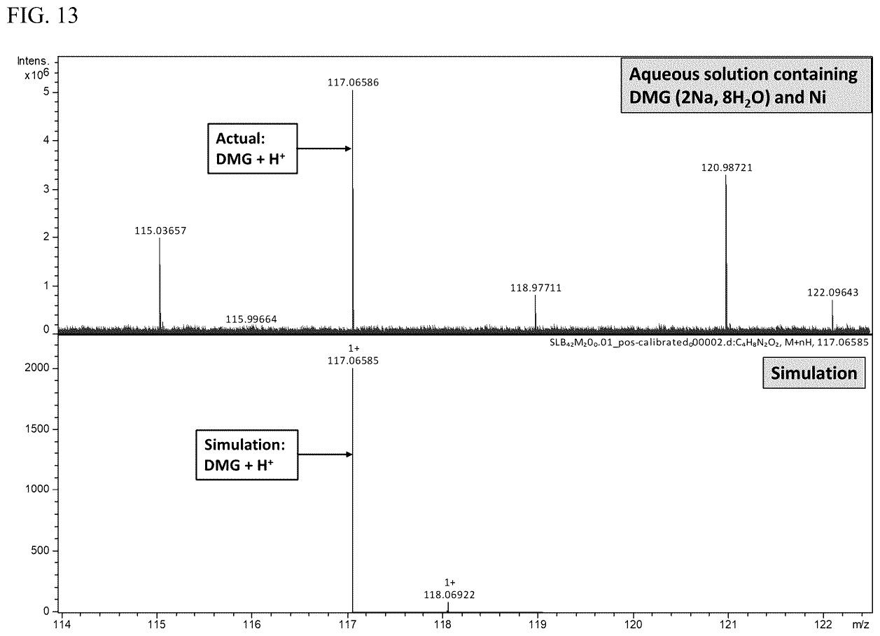

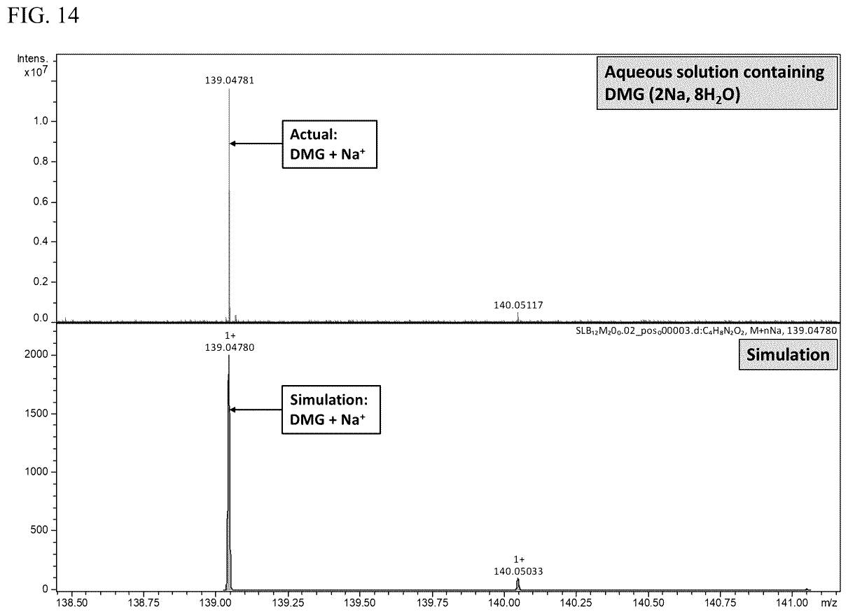

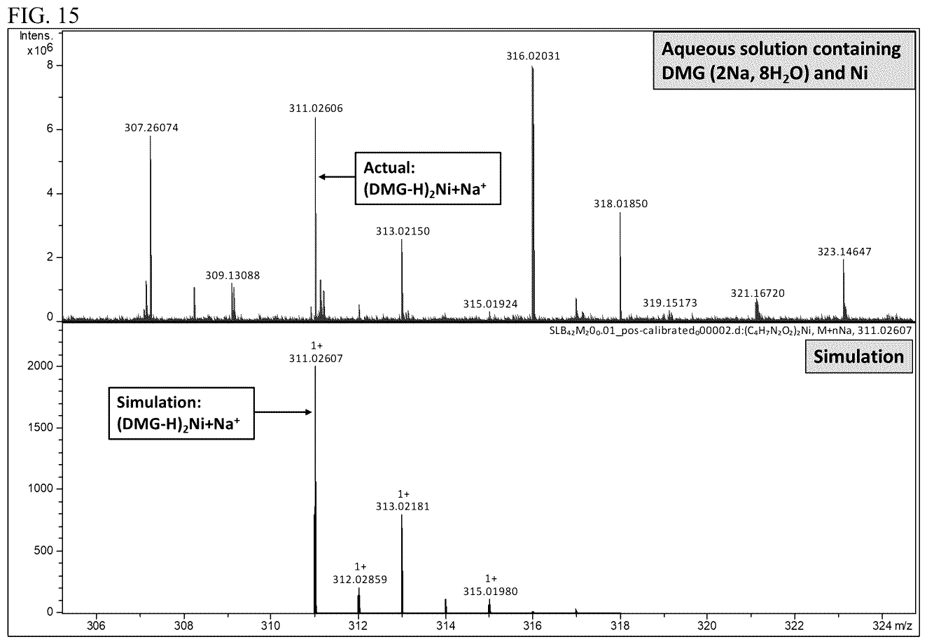

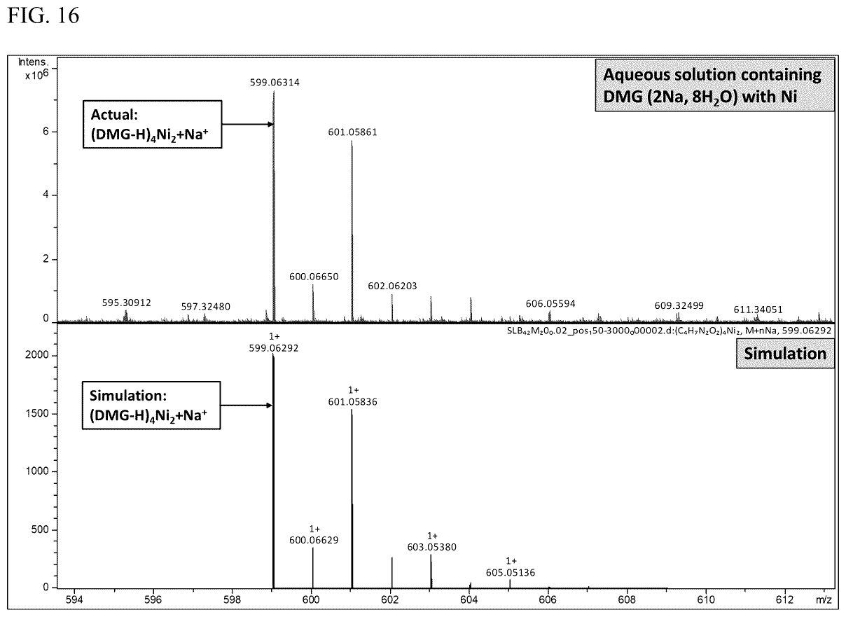

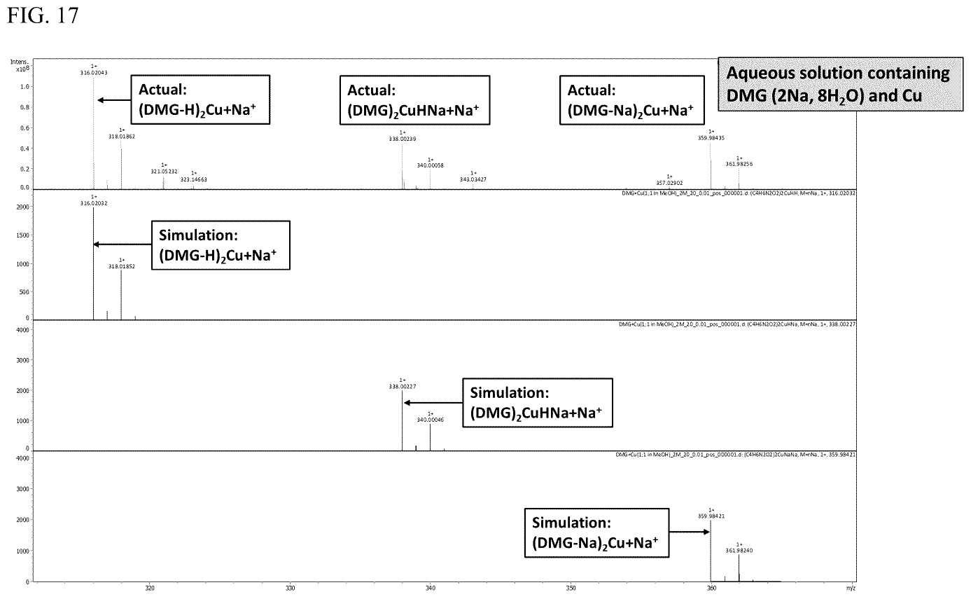

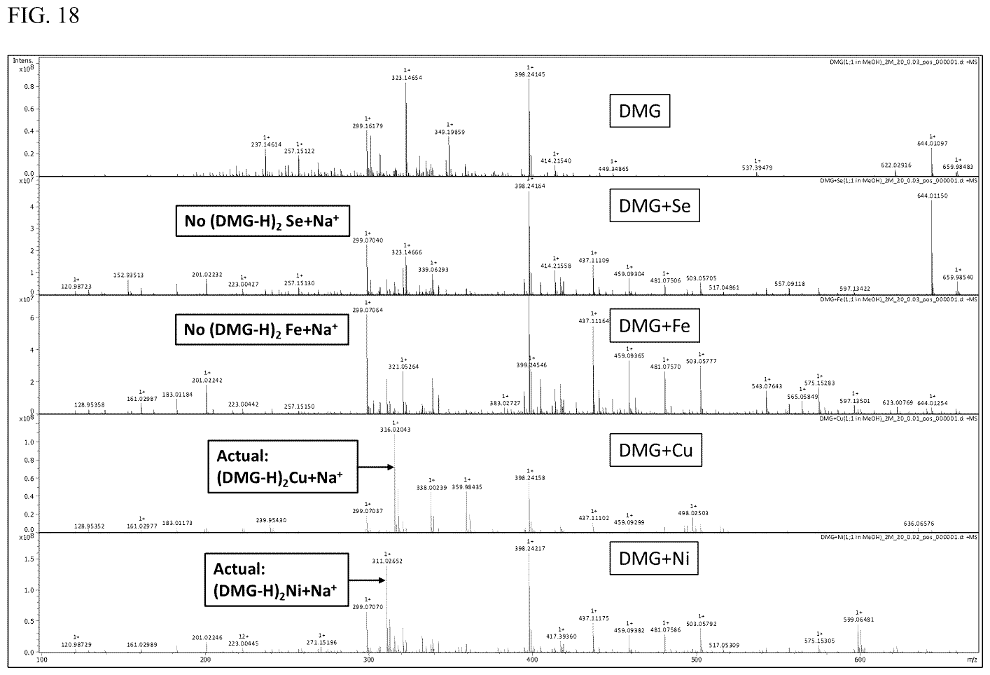

DMG-metal complexes can be detected by FTICR-MS. Aqueous solutions containing only DMG, or DMG in combination with Ni, Cu, Fe, Se or Zn salts were analyzed using FTICR-MS. In absence of added metal, two monomeric isoforms were detected, corresponding to either DMG, H⁺ (117.06585 m/z) or DMG, Na⁺ (139.04780 m/z) (FIG. 12-FIG. 18). In presence of Ni, complexes consisting of two DMG and one Ni, with either H⁺ (289.04412 m/z) or Na⁺ (311.02607 m/z) were detected. This was expected, as two DMG are required to chelate one Ni (FIG. 1). Surprisingly, FTICR-MS analysis of a DMG-Ni aqueous solution further revealed the presence of [DMG]₄—(Ni)₂ complexes, mostly in the Na⁺ form (599.06292 m/z) (FIG. 12-FIG. 18). While [DMG]₂—Cu complexes were identified, only monomeric DMG (H⁺ or Na⁺) was observed in presence of Fe, Zn or Se; no dimeric or tetrameric DMG-Se or DMG-Zn complexes could be detected, suggesting DMG does not coordinate with Fe, Zn or Se (FIG. 12-FIG. 18).

Analysis of DMG in brain samples using FTICR-MS and NMR. The fact that DMG inhibits Aβ₄₀ peptide aggregation in vitro suggests it might be able to do the same in vivo, however DMG would first need to cross the blood brain (BBB). To determine whether DMG can localize to the mouse brain, we used FTICR-MS (see above) and Nuclear Magnetic Resonance (NMR). NMR was successfully used to detect DMG in the livers of mice subjected to daily oral doses (6.1 mg) of aqueous DMG for 3 days (Benoit et al. Sci Rep 9, 13851 (2019)). In the present study, the same treatment was administered (e.g. one daily oral delivery for 3 days), brain samples were processed and analyzed by NMR and FTICR-MS and compared to brain samples from (no DMG) control mice. Unfortunately, both methods failed to identify DMG (whether by itself or metal-chelated) in brain samples.

### Experimental Procedures

Chemicals. The water-soluble form (2Na, 8H₂O) of DMG was used in this study (ref #40400, Honeywell-Fluka, Muskegon, MI, USA). All metals (CuSO₄, FeSO₄, Na₂SeO₃, NiCl₂, NiSO₄, ZnSO₄) were from Sigma-Aldrich (Saint Louis, MO, USA).

Amyloid Beta metal analysis. Metal levels for 20 elements (Li, Be, Al, V, Cr, Mn, Fe, Co, Ni, Cu, Zn, As, Se, Rb, Sr, Cd, Cs, Ba, Pb, U) were determined by inductively coupled plasma mass spectrometry (ICP-MS). Briefly, 0.5 mg of lyophilized human recombinant Ab₄₀ peptide (expressed in E. coli, purified and manufactured by rPeptides, Watkinsville, GA) was resuspended in ultrapure water to a final concentration of 5 mg/mL, digested overnight with concentrated trace metal grade nitric acid, heated for 2 h at 95° C. and subjected to ICP-MS using a Thermo X-Series II ICP-MS (Center for Applied Isotope Studies, University of Georgia, Athens, GA). The same treatment and analysis were performed on three other kit components: TBS 10× (reaction buffer), Thioflavin stock (400 μM) and NaOH (10 mM, used to resuspend the Ab₄₀ peptide).

Amyloid Beta aggregation. The effect of metals, DMG, and/or pH on human recombinant aggregation was monitored using a thioflavin T (ThT)-based kit, following the manufacturer's recommendation (kit #A-1180-1, rPeptides, Watkinsville, GA, USA). This kit contained human recombinant Aβ₄₀ peptide (>97% pure, as determined by manufacturer's HPLC) with the following sequence DAEFRHDSGYEVHHQKLVFFAEDVGSNKGAIIGLMVGGVV (SEQ ID NO:1), as provided by the manufacturer. Briefly, standard assays were conducted in triplicate in black polystyrene 96-well plates, in presence of Tris Buffer Saline (TBS) pH 7.4, or TBS pH 8.5, or 2-(N-morpholino)ethanesulfonic acid (MES) buffer saline pH 6.5, Th-T (20 or 40 μM), and human recombinant Aβ₄₀ peptide (25 or 40 μM), with or without DMG (100, 500, or 1000 μM), CuSO₄, NiSO₄ or ZnSO₄ (10, 25, or 100 μM). The aggregation of Aβ₄₀, was shown by the increase in fluorescence (lex=440 nm/lem=485 nm) over time, was followed for 60 min or 120 min, with reading every three or five min, using a Synergy MX reader (Biotek, Winooski, VT). A ThT-only background control (no Aβ₄₀ peptide) was included in triplicate in all experiments and subtracted from all readings. The aggregation rate, defined as the increase in fluorescence per min (RFU/min), was calculated by using the formula [A(440,485) at T40 min)-A(440,485) at T20 min]/20. These time points (20 to 40 min) were chosen because they constantly displayed the best linearity in every assay. Results shown are expressed as (mean and standard deviation of) percentages; they represent the ratio of aggregation rate (RFU/min) for a given (DMG, metal) condition, compared to the aggregation rate obtained for the control (Aβ₄₀ peptide, no DMG, no metal added, set as 100% for each experiment).

Isothermal Titration Calorimetry. Binding assays of Aβ₄₀ peptide and Ni or DMG were performed using a Nano ITC calorimeter (TA instruments, New Castle, DE). Briefly, 1 mg of lyophilized Aβ₄₀ peptide (#A-1157-2, rPeptides) was resuspended with 1% NH₄₀H to a concentration of 250 μM, sonicated for 15-20 sec, before being diluted to a final concentration of 20 μM Aβ₄₀ using ddH₂O and TBS 10×, pH 7.4 (working buffer: NH₄₀H 0.2%, TBS 1×, pH 7.4 (“NTBS”)). A volume of 500 μL was loaded onto the ITC sample cell, and the injection syringe was filled with 50 μL of either NTBS buffer (control), 1 mM NiSO₄, or 1 mM DMG. All samples were degassed for 15 min at 25° C. before use. Titration was initiated using a program for 20 injections (2.38 μL each, every 5 min) with continuous stirring (350 rpm) at constant temperature (25° C.). ITC data were analyzed using NanoAnalyze 1.2 software (TA Instruments). Data obtained with the control experiment (Aβ₄₀ peptide in sample cell, buffer in syringe) were subtracted from each experiment to account for any injection-related heat change. The Ni-Aβ₄₀ experiment was done in triplicate, with a representative data set shown in figures.

Analysis of DMG and DMG-metal complexes in commercial preparations. Aqueous solutions of DMG (0.5 mg/mL or 1.6 mM), with or without Cu, Fe, Ni, Se or Zn solutions (0.16 mM each) were analyzed by Fourier Transform Ion Cyclotron Resonance Mass Spectrometry (FTICR-MS), using a Bruker Solari X ESI/MALDI-12T FT-ICR high precision mass spectrometer (Proteomics and Mass Spectrometry Facility, University of Georgia). The pH of all aqueous DMG solutions, with or without metal, was approximately 11. All samples were mixed (1:1) with methanol prior to injection.

Detection of DMG and DMG-metal complexes in mouse brains. A group of 6 (C57/BL) mice was used for this experiment: 3 mice were given 0.2 mL of 100 mM DMG (˜6.1 mg) every day for three days and 3 mice were used as (no DMG) controls. Mice were euthanized by CO₂ asphyxiation and cervical dislocation. Brains were quickly removed and frozen at −80° C. Upon thawing, brains were cut into pieces and homogenized in 2 mL sterile deionized water, incubated for 1 h at 90° C., sonicated for 20 sec and spun down (16,800×g for 6 min). Supernatants were passaged through a 0.45 m filter unit and analyzed by FTICR-MS (see above) and Nuclear Magnetic Resonance (NMR), as previously described (Benoit et al. Sci Rep 9, 13851 (2019)).

### Example 3—Dimethylglyoxime Inhibition of Biofilms

### Methods

Growth conditions. Salmonella enterica serovar Typhimurium (S. Typhimurium) ATCC 700408 and Klebsiella pneumoniae ATCC BAA2472 were grown overnight in Luria-Bertani (LB) broth aerobically at 37° C. with shaking. Helicobacter pylori 43504 was grown on Brucella agar plates supplemented with 10% defibrinated sheep blood (BA) under microaerophilic conditions (4% 02, 10% CO₂, and 86% N₂) at 37° C.

Biofilm inhibition assay. S. Typhimurium and K. pneumoniae cultures were adjusted to an optical density (OD₆₀₀) of 0.1 in fresh LB broth. H. pylori culture was adjusted to an OD₆₀₀ of 0.15 in brain heart infusion (BHI) supplemented with 0.4% O-cyclodextran. S. Typhimurium, K. pneumoniae, and H. pylori cultures at the above cell densities were added to non-tissue culture treated polystyrene 96 well plates (Falcon) along with dimethylglyoxime disodium salt hydrate (Na₂DMG) at various concentrations. Plates containing S. Typhimurium and K. pneumoniae were incubated at 37° C. aerobically without shaking for 18 hours. Plates containing H. pylori were incubated at 37° C. microaerobically without shaking for 48 hours. After incubation with (Na₂DMG) a crystal violet biofilm assay was performed as previously described with modifications (Kwasny et al. 2010. Current Protocols in Pharmacology 50:13A. 8.1-13A. 8.23). Briefly, supernatant was removed from the wells. Next, adherent cells were gently washed with phosphate-buffered saline (PBS) and allowed to air dry for 15 minutes. Cells were then heat fixed for one hour at 60° C. before staining with 0.5% crystal violet for 5 minutes. After the crystal violet was thoroughly washed from the wells with distilled deionized water, cells were air dried for 15 minutes. Acetic acid (33%) was then added to the wells to solubilize the crystal violet. The crystal violet-acetic acid solution was transferred to a clean 96 well plate and the OD₆₀₀ was measured.

Antibiofilm assay. H. pylori culture at OD₆₀₀=0.15 was added to a non-tissue culture treated polystyrene 96 well plate (Falcon) and incubated at 37° C. micro-aerobically without shaking for 48 hours. After the initial incubation, the supernatant was removed from the wells and BHI+0.4% β-cyclodextran was added back to the wells with or without Na₂DMG at various concentrations. The plate was then incubated an additional 24 hours. Then, the crystal violet biofilm assay was performed as above.

### Results

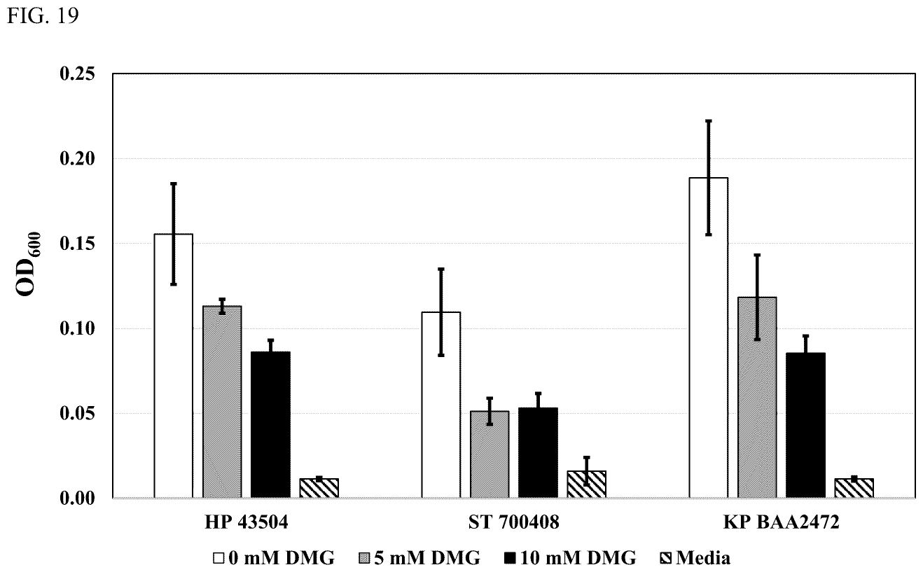

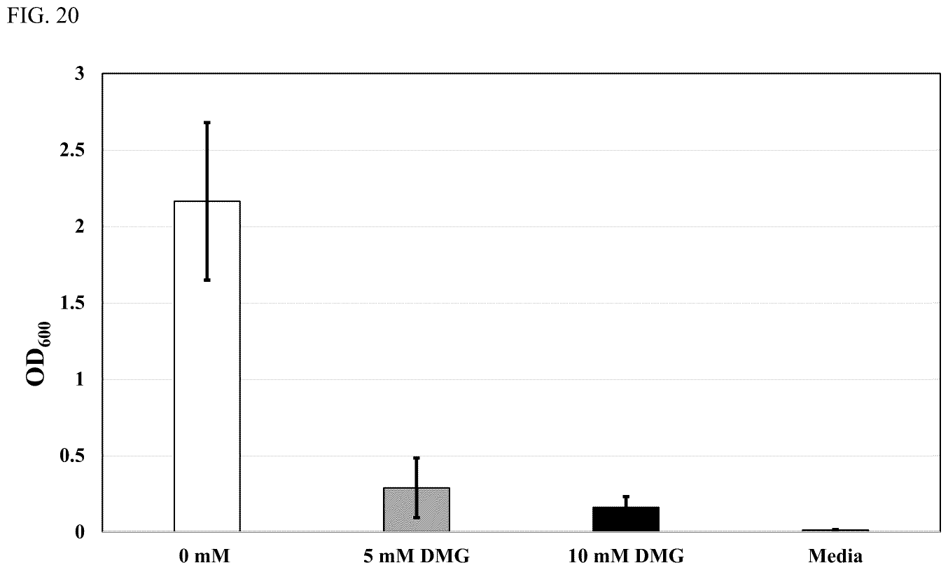

In all three species tested (S. Typhimurium, K. pneumoniae, and H. pylori), DMG was able to inhibit biofilm formation (FIG. 19). Additionally, DMG was able to counteract an established (48 hours of growth) H. pylori biofilm (FIG. 20). These experiments provide evidence for promising antibiofilm properties of DMG.

### Example 4—Effect of DMG (Alone or in Combination with Metal Ions) on Campylobacter Species

This Example shows that DMG (at millimolar levels) is bactericidal against Campylobacter concisus. This Example further shows that while DMG (at millimolar levels) is bacteriostatic against Campylobacter jejuni, DMG at millimolar levels is bactericidal against C. jejuni when combined with micromolar levels of copper (Cu²⁺).

C. concisus has been found throughout the entire human oral-gastrointestinal tract. The bacterium is associated with various ailments and diseases, such as gingivitis, periodontitis, inflammatory bowel disease, including Crohn's disease. There is no known animal reservoir.

C. jejuni is one of the most common causes of food poisoning in Europe and in the United States. The CDC estimates a total of 1.5 million infections every year and the European Food Safety Authority estimates approximately nine million cases of human campylobacteriosis per year in the European Union. People are usually infected by eating raw or undercooked poultry, or eating something that touched the raw or undercooked poultry. People may also be infected by eating seafood, meat, and produce; by contact with animals (C. jejuni is commonly found in animal feces); or by drinking untreated water. Food poisoning caused by Campylobacter species can be severely debilitating, however it is rarely life-threatening. Nevertheless, it can subsequently lead to Guillain-Barre syndrome (GBS), an auto-immune disease targeting the nerves and eventually causing paralysis.

Fluoroquinolone-resistant Campylobacter species are classified as “Priority 2: high” in the WHO's list of MDR pathogens (see Table 1A). All Campylobacter species have at least one Ni-containing hydrogenase (for instance C. jejuni), and some have two (for instance C. concisus). In the case of C. concisus, one of the hydrogenase complexes has been shown to be essential (Benoit et al. 2018 Sci. Rep. 8(1):14203). Hence, inhibiting the Ni-containing catalytic site of C. concisus (with DMG) was expected to have inhibitory effects on C. concisus growth and survival. As further described, below, however, the combined effect of DMG and Cu² on C. jejuni was unexpected.

### Methods

Growth conditions. C. concisus (strain 13826, ATCC BAA-1457) was routinely grown on Brucella agar supplemented with 10% defibrinated sheep blood (BA plates) or Brain Heart Infusion (BHI) plates supplemented with 10% fetal calf serum (FCS), under H₂-enriched microaerobic conditions (10% H₂, 5% CO₂, 2-10% 02, balance N₂) at 37° C. C. jejuni (strain 81-176, ATCC BAA-2151) was either grown on BA plates or on BHI plates under microaerobic conditions (10% CO₂, 4% 02, 86% N₂) at 37° C. DMG (sodium salt, octahydrate) was used by itself or in combination with various divalent cations (Cobalt (II), CoCl₂; Copper(II), CuSO₄; Manganese(II), MnSO₄; Nickel(II), NiSO₄; Zinc(II), ZnSO₄)

Growth inhibition assays: solid (Petri dish) plates experiments. C. concisus or C. jejuni cells were grown as described above, harvested, and resuspended in sterile NaCl (8 g/L) to an optical density at 600 nm (OD₆₀₀) of 0.1 or 1; an OD₆₀₀ of 1 corresponds approximately to 1×10⁹ cells/mL to 5×10⁹ cells/mL (for C. concisus) and 5×10⁹ cells/mL to 1×10¹⁰ cells/mL (for C. jejuni). Cells were serially (10-fold) diluted from 10⁻¹ to 10⁻⁶ or 10⁻⁷ in NaCl (8 g/L) and 5 μL of each dilution was spotted on BA plates (for C. concisus) or BHI plates (for C. jejuni), containing various concentrations of DMG (0.5, 1, 2.5 or 5 mM), with or without various added metals (CoCl₂, CuSO₄, MnSO₄, NiSO₄, ZnSO₄) at the following final concentrations: 1 μM, 5 μM, 10 μM, 20 PM, 50 μM, 100 μM, 500 M. Plates with metals only were also used as controls. Colony-forming units (CFUs) were counted after 24 hours of growth at 37° C., under atmospheric conditions described above. The number of CFUs for each (DMG and metal) condition was compared to the number of CFUs obtained for the control condition (defined as plain BA, plain BHI-FCS, or plain BHI medium, without any DMG or added metal). Also, the size of the colonies was monitored. DMG concentrations were considered bacteriostatic when the number of CFUs was equal to the number of CFUs of the control, but the size of each CFU was significantly smaller compared to the control. DMG concentrations were considered bactericidal when the number of CFUs was at least three logs less than the number of CFUs obtained for the control.

Growth inhibition assays: liquid (96-well) plates experiments. This type of experiment can only be conducted with C. jejuni since the gas requirement for C. concisus (H₂-enriched microaerobic) is not compatible with the use of 96-well plates. C. jejuni cells were grown as described above, harvested, and resuspended in sterile NaCl (8 g/L) to an OD₆₀₀ of 0.1, corresponding approximately to 5×10⁸ to 1×10⁹ cells/mL. A checkboard type of loading plan allows for screening of up to eight DMG concentrations (0 mM, 0.062 mM, 0.125 mM, 0.25 mM, 0.5 mM, 1 mM, 2 mM, and 4 mM) against 11 metal ions concentrations (0 μM, 2 μM, 4 μM, 8 μM, 16 μM, 32 μM, 64 μM, 128 μM, 256 μM, 512 μM, and 1024 μM). Wells were serially (2-fold) diluted in each direction (horizontal and vertical) to introduce all combinations. Wells with no DMG or metal compounds served as (positive) growth controls, and wells with no bacteria served as background (negative growth) controls. A typical protocol and loading plan are described in Table 7. The starting inoculum was at OD₆₀₀ of 0.05, which corresponds approximately to 2.5×10⁸ cells/mL to 5×10⁸ cells/mL. After 24 hours incubation under microaerobic conditions at 37° C., OD₆₀₀ was recorded using a 96 well-plate reader (Microtek). The minimal inhibitory concentration (MIC) for DMG (or metal) is defined as the lowest concentration that inhibits cell growth (for example, OD₆₀₀ at T=24 hours is the same or lower than 0.05) The minimal bactericidal concentration (MBC) was determined as follows: at the end of the MIC assay (24 hours), 5 μL of each well showing no or low growth was spotted on BHI and incubated at 37° C. overnight. Resulting growth (or lack of growth) was examined after overnight culturing; the lowest concentration that inhibits 99.9% of the original culture was defined as MBC. MIC and MBC were determined based on tests (plates) done in triplicate.

### Results

### Effect of DMG on C. Concisus: Millimolar Levels of DMG are Bactericidal.

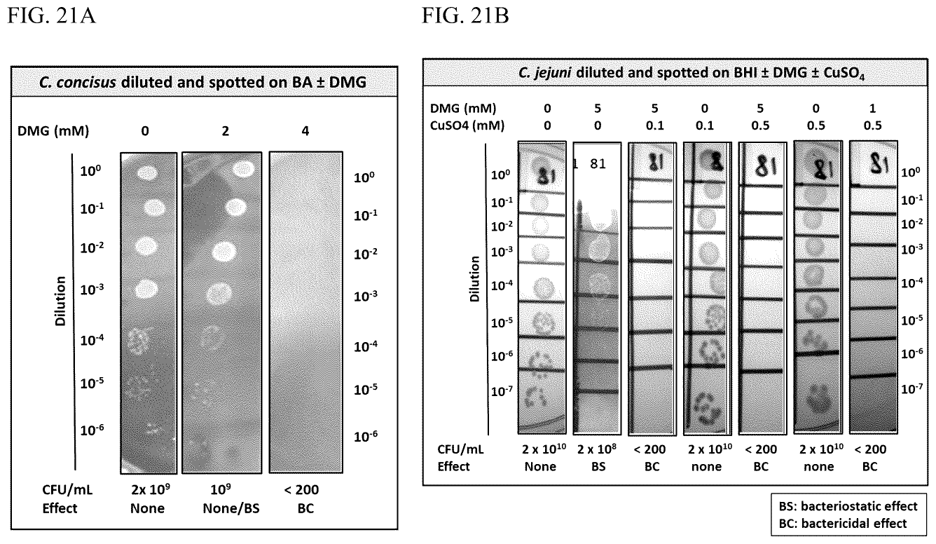

C. concisus recoveries on solid plates. After dilution and spotting on BA plates with no DMG or 2 mM DMG or 4 mM DMG, cells were incubated under H₂-enriched microaerobic conditions for 24 hours at 37° C. and CFUs were counted (FIG. 21A, for an example). The number of CFUs on BA plates supplemented with DMG 2 mM were equal or slightly lower than the number of CFUs on the control plates (no DMG), however the size of the colonies was smaller, suggesting the DMG concentration (2 mM) is bacteriostatic. There was no CFU detected on BA supplemented with DMG 4 mM (detection limit: 200 CFUs), indicating DMG at 4 mM is bactericidal for C. concisus under these experimental conditions.

Effect of DMG on C. jejuni: Millimolar Levels of DMG are Bacteriostatic, but Addition of Micromolar Levels of Copper (II) Renders Millimolar Levels of DMG Bactericidal Towards C. Jejuni.

C. jejuni recoveries on solid plates. After dilution and spotting on BHI plates with no DMG, or various concentrations of DMG with or without supplemental CuSO₄ or other metals (see methods), cells were incubated under microaerobic conditions for 24 hours at 37° C. and CFUs were counted (FIG. 21). The number of CFUs on BHI plates supplemented with 5 mM DMG were equal or slightly lower than the number of CFUs on the control plates (having no DMG), however the size of the colonies was smaller, suggesting the DMG concentration (5 mM) is bacteriostatic. When copper(II) (CuSO₄) at the following final concentrations: 1 μM, 5 μM, 10 μM, 20 μM, 50 μM, 100 μM, 500 μM) was added to 5 mM DMG plates, there was no detectable growth on the plates (detection limit: 200 CFUs), indicating all of the above combinations were bactericidal for C. concisus under these experimental conditions (FIG. 21).

The bactericidal effect of the DMG/metal combination is specific to Cu(II). For instance, while a DMG (2 mM)/Cu (0.5 mM) combination is bactericidal, there was no noticeable effect on C. jejuni growth when 0.5 mM of CoCl₂, MnSO₄, NiSO₄, or ZnSO₄ was added to the medium in combination with 2 mM DMG.

C. jejuni liquid (96-well checkboard assays) growth inhibition experiments. After 24 hours incubation under microaerobic conditions at 37° C., the OD₆₀₀ was recorded in each well using a 96 well-plate reader. Table 8 shows an example of results obtained with various DMG and CuSO₄ concentrations (three plates combined). In this case, the minimal inhibitory concentration (MIC), as defined in the Methods section, corresponds to the following combinations of DMG (in mM) and CuSO₄ (in M): 4 and 8; 2 and 64; 1 and 256 (see Table 9).

After the OD₆₀₀ was recorded, 5 μL of each well showing no or low growth was spotted on BHI and incubated at 37° C. overnight to determine the minimal bactericidal concentration (MBC), as defined in the Methods section.

As observed with the solid plate experiment, the bactericidal effect of DMG/metal combination is specific to Cu(II). Other metals did not confer the same potency in combination with DMG, therefore the DMG/Cu combination is the most efficient (e.g., bactericidal) against C. jejuni. Table 9 summarizes the MIC/MBC obtained for C. jejuni, with DMG and either CoCl₂, MnSO₄, NiSO₄, or ZnSO₄.

TABLE 7
C. jejuni 81-176 against DMG and CuSO₄, checkerboard strategy.

| | | Final CuSO₄ concentration (μM) | | | | | | | | | | | |
|---|---|---|---|---|---|---|---|---|---|---|---|---|---|
| Final | | 0 | 2 | 4 | 8 | 16 | 32 | 64 | 128 | 256 | 512 | 1024 | 1024 |
| DMG (mM) | | 1 | 2 | 3 | 4 | 5 | 6 | 7 | 8 | 9 | 10 | 11 | 12 |
| 4 | A | no Cu | | | | | | | | | | | no cell |
| 2 | B | no Cu | | | | | | | | | | | no cell |
| 1 | C | no Cu | | | | | | | | | | | no cell |
| 0.5 | D | no Cu | | | | | | | | | | | no cell |
| 0.25 | E | no Cu | | | | | | | | | | | no cell |
| 0.125 | F | no Cu | | | | | | | | | | | no cell |
| 0.062 | G | no Cu | | | | | | | | | | | no cell |
| 0 | H | no Cu/ | no | no | no | no | no | no | no | no | no | no | Blank |
| | | no DMG | DMG | DMG | DMG | DMG | DMG | DMG | DMG | DMG | DMG | DMG | |

1 Add 100 uL of MH everywhere (A1-H12)
2 Add 100 uL of 16 mM DMG (240 ul 200 mM DMG•8H2O in 2760 uL MH) in rows A1-A12
3 Serially (2-fold) dilute DMG from row A (A1-A12) to row G (G1-G12). Discard 100 uL after row G.
4 Volume should be 100 uL everywhere and DMG concentration should be 8 mM (row A) to 0.125 mM (row G), no DMG in row H
5 Add 100 uL of 4.096 mM CuSO4 (61.5 uL 200 mM CuSO4 in 3 mL MH) in columns 11 (A11-H11) and 12 (A12-H12). Column 12: no cell control (metal-only OD₆₀ [symbol]
6 Serially (2-fold) dilute metal from column 11 (A11-H11) to column 2 (A2-H2). Discard 100 uL after column 2.
7 Volume: 100 uL everywhere, except in 12 (200 uL); CuSO4 concentration: 2048 uM (Column 12) to 2 uM (Column 2), no Cu in column 1.
8 Add 100 uL of bacteria (C. jejuni) at OD 0.1 everywhere except in column 12. Starting OD should be 0.05 everywhere.
9 Final concentrations: DMG concentration range from 4 to 0.062 mM, CuSO4 conc. range from 1.024 mM to 0.002 mM (1,024 uM to 2 uM)
10 Incubate C. jejuni in CO₂ incubator (4% O₂, 10% CO₂, balance N₂) for 24 h
11 Measure OD₆₀₀ at 24 h. Blank with A12. Use Column 12 to withdraw Cu-and DMG-Cu absorbance. [symbol]
indicates data missing or illegible when filed

TABLE 8

| 3 plates combined | | | | | Final CuSO₄ concentration (uM) | | | | | | | | No cell |
|---|---|---|---|---|---|---|---|---|---|---|---|---|---|
| Final | | 0 | 2 | 4 | 8 | 16 | 32 | 64 | 128 | 256 | 512 | 1024 | 1024 |
| DMG (mM) | | 1 | 2 | 3 | 4 | 5 | 6 | 7 | 8 | 9 | 10 | 11 | 12 |
| 4 | A | 0.19 | 0.26 | 0.15 | 0.04 | 0.00 | 0.00 | 0.01 | 0.01 | 0.02 | 0.02 | 0.04 | 0.04 |
| 2 | B | 0.24 | 0.34 | 0.25 | 0.26 | 0.24 | 0.17 | 0.01 | 0.01 | 0.01 | 0.02 | 0.04 | 0.04 |
| 1 | C | 0.20 | 0.27 | 0.23 | 0.21 | 0.37 | 0.24 | 0.19 | 0.10 | 0.02 | 0.02 | 0.04 | 0.04 |
| 0.5 | D | 0.22 | 0.31 | 0.25 | 0.23 | 0.34 | 0.21 | 0.19 | 0.20 | 0.29 | 0.14 | 0.09 | 0.04 |
| 0.25 | E | 0.26 | 0.31 | 0.24 | 0.37 | 0.33 | 0.25 | 0.22 | 0.24 | 0.24 | 0.27 | 0.30 | 0.04 |
| 0.125 | F | 0.25 | 0.31 | 0.27 | 0.31 | 0.33 | 0.26 | 0.23 | 0.24 | 0.27 | 0.34 | 0.47 | 0.04 |
| 0.062 | G | 0.31 | 0.37 | 0.31 | 0.33 | 0.34 | 0.24 | 0.22 | 0.24 | 0.35 | 0.34 | 0.47 | 0.04 |
| 0 | H | 0.23 | 0.29 | 0.23 | 0.31 | 0.38 | 0.26 | 0.27 | 0.23 | 0.38 | 0.28 | 0.43 | 0.04 |

TABLE 9
MIC/MBC for DMG-metal combinations, C. jejuni strain 81-176

| | DMG concentration (mM) | | | | |
|---|---|---|---|---|---|
| Metal | 0 | 0.5 | 1 | 2 | 4 |
| Co²⁺ | >1024/>1024 | >1024/>1024 | >1024/>1024 | 512/1024 | 256/512 |
| Cu²⁺ | >1024/>1024 | 1024/>1024 | 256/512 | 64/128 | 8/16 |
| Ni²⁺ | >1024/>1024 | >1024/>1024 | >1024/>1024 | >1024/>1024 | >1024/>1024 |
| Zn²⁺ | 512/>1024 | 512/>1024 | 512/>1024 | 512/>1024 | 512/>1024 |

First number, bold: MIC (μM)/
second number, italic: MBC (μM)

### Example 5

This Example shows that DMG inhibits C. jejuni colonization in some chickens in the presence of added copper. This Example further shows that DMG inhibits C. jejuni colonization in some chickens in the absence of added copper.

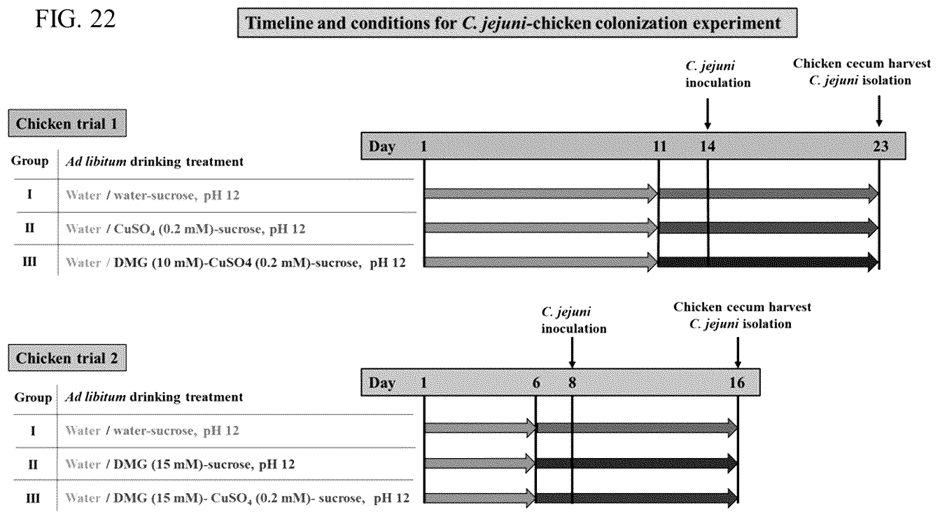

Since chickens are natural hosts for C. jejuni, and chicken meat is one of the main sources of campylobacteriosis in humans, it was important to test the efficacy of DMG alone or in combination with copper in this animal model. Two independent trials were conducted, as described in FIG. 22. In both trials, the treatment was administered through ad libitum drinks, starting two to three days before C. jejuni inoculation (with strain NCTC 11168), and going through the end of each experiment. In the first trial, the efficacy of DMG (10 mM) combined with CuSO₄ (0.2 mM), was compared to that of a CuSO₄ (0.2 mM)-only control and a water-only control (FIG. 22). Since addition of DMG both increased the pH (˜12) and made drinking solutions less palatable (as observed during preliminary experiments, where chickens refused to drink and became dehydrated), sucrose (10 g/L) was added in all solutions, and the pH of all drinking solutions was adjusted to pH 12 in both trials to account for any pH-related effect. Nine days after inoculation with C. jejuni, chickens were euthanized, and their ceca were isolated for bacterial count. Results from the first trial showed a 3-fold to 5-fold decrease in bacterial count in the cecum of the Cu-DMG group compared to the water-only control and the copper-only control, respectively (Table 10). Importantly, C. jejuni was not recovered in 33% of the chickens belonging to the Cu-DMG treated group (n=6), suggesting that the treatment abolished C. jejuni colonization of the cecum, in agreement with the in vitro bactericidal effect described earlier in Example 4. In a second trial, the efficacy of DMG (15 mM) combined with CuSO₄ (0.2 mM) to that of DMG only (15 mM) and a water control (all solutions with glucose, at pH 12) was compared. The treatment was administered two days before inoculation with C. jejuni, and chicken ceca were harvested eight days after inoculation. Ad libitum drinking of DMG alone resulted in approximately 3-fold less bacterial colonization of the cecum compared to the water only control; one chicken in this group (DMG only) appeared to be C. jejuni free (Table 10). Treatment with DMG and copper resulted in approximately 3.5-fold decrease in bacterial burden compared to the water control. Taken together, these results suggest that millimolar concentrations of DMG, by itself or in combination with micromolar concentration of CuSO₄, can lower and, in some cases, even fully inhibit C. jejuni colonization of the chicken cecum.

TABLE 10
C. jejuni colonization of chickens subjected to various drinking treatments.

| | Chickens | | | C. jejuni |
|---|---|---|---|---|
| Ad libitum | Total | Colonized | Cj-free** | Average CFUs per |
| oral treatment | (n=) | (n=) | (n=) | g of cecum (×10⁸) |
| Experiment 1 | | | | |
| Water | 5 | 5 | 0 | 3.56 |
| CuSO₄ (0.2 mM) | 4 | 4 | 0 | 5.95 |
| CuSO₄ (0.2 mM) | 6 | 4 | 2 | 1.12 |
| DMG (10 mM) | | | | |
| Experiment 2 | | | | |
| Water | 6 | 6 | 0 | 6.73 |
| DMG (15 mM) | 7 | 6 | 1 | 2.38 |
| CuSO₄ (0.2 mM) | 5 | 5 | 0 | 1.99 |
| DMG (15 mM) | | | | |

* ad libitum oral treatment: chickens had free access to the indicated drinking treatment, starting two days before C. jejuni inoculation, and until the day of harvest (9 days p.i. for experiment 1; 8 days p.i. for experiment 2).
**Cj-free: no C. jejuni was recovered from cecum content (detection limit = 200 CFUs per g of cecum).

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1.-8. (canceled)

9. A method for administering to a subject a composition comprising:
a chelator wherein the chelator comprises soluble dimethylglyoxime (DMG); and
a carrier.

10. The method of claim 9, wherein the subject comprises a human or an animal.

11. The method of claim 10, wherein the animal comprises a chicken.

12. The method of claim 9, wherein the composition further comprises copper.

13. The method of claim 9, wherein the subject is infected with a pathogen or susceptible to infection by a pathogen.

14. The method of claim 13, wherein the pathogen comprises a pathogen that comprises a nickel-containing enzyme, a fungus that comprises a nickel-containing enzyme, or a non-fungal eukaryotic pathogen that comprises a nickel-containing enzyme.

15. The method of claim 13, wherein the pathogen comprises a multi-drug resistant pathogen.

16. The method of claim 13 wherein the pathogen comprises Acinetobacter baumannii, Enterococcus faecium, Escherichia coli, Helicobacter pylori, Haemophilus influenzae, Neisseria gonorrhoeae, Streptococcus pneumoniae, a Campylobacter species, an Enterobacter species, a Klebsiella species, a Morganella species, a Proteus species, a Providencia species, a Pseudomonas species, a Salmonella species, a Serratia species, a Shigella species, or a Staphylococcus species, or a combination thereof,
Cryptococcus neoformans, Cryptococcus gattii, Coccidioides posadasii, Histoplasma capsulatum, or Paracoccidioides brasiliensis, or a combination thereof; or
Pythium insidiosum, Leishmania major, Leishmania donovani, or Trypanosoma cruzi, or a combination thereof.

17. The method of claim 9, wherein the subject is suffering from or susceptible to a disease associated with Amyloid-β peptide aggregation.

18. The method of claim 17, wherein the disease is Alzheimer's disease, Down Syndrome, or both.

19.-20. (canceled)
